(19) United States
(12) Patent Application Publication
(10) Pub. No.: US 2006/0281675 A1
(43) Pub. Date: Dec. 14, 2006

(54) Somatogenic therapy using a 20kda placental variant of growth hormone

(75) Inventors: Peter Gluckman, Auckland (NZ); Robert Stewart Gilmour, Little Abington (GB); Mark Hedley Vickers, Auckland (NZ); Bernhard Hermann Heinrich Breier, Auckland (NZ)

Correspondence Address: FLIESLER MEYER, LLP, FOUR EMBARCADERO CENTER, SUITE 400, SAN FRANCISCO, CA, 94111, US

(73) Assignee: NEUREN PHARMACEUTICALS LIMITED, Auckland (NZ)

(21) Appl. No.: 10/568,573

(22) PCT Filed: Aug. 19, 2004
(86) PCT No.: PCT/US04/27187

§ 371 Date: Aug. 10, 2006

Related U.S. Application Data
(60) Provisional application No. 60/496,970, filed on Aug. 20, 2003.

(51) Int. Cl. A61K 38/27

(52) U.S. Cl. 514/12

(57) ABSTRACT

Embodiments of the present invention provide improved methods of treating conditions requiring human growth hormone (hGH) therapy, whereby the beneficial effects of hGH such as growth promotion and lipolysis are retained and unwanted properties are reduced or eliminated. In particular, it is directed at a method of treatment whereby the lactogenic side effects of hGH treatment are reduced. Said enhanced method includes the use of a growth hormone variant; 20 kDa hGH-V in the treatment of conditions that are currently treated with hGH or that have the potential to be treated with hGH.

### RELATED APPLICATION

This application claims priority to U.S. Provisional Patent Application Ser. No. 60/496,970, filed Aug. 20, 2003, Peter Gluckman and Stewart Gilmour, Inventors, titled “Enhanced Growth Hormone Therapy Using a 20 kDa Placental Variant of Growth Hormone” (Attorney Docket No: ERNZ 1016 US0 DBB), incorporated herein fully by reference.

### FIELD OF THE INVENTION

This invention pertains to conditions and diseases for which growth hormone is a desirable method of treatment. In particular, the invention pertains to the treatment of such conditions and diseases using variants of growth hormone. More particularly, the invention pertains to the treatment of such conditions and diseases using a 20 kiloDalton placental growth hormone variant (“20 kDa hGH-V”).

### BACKGROUND

There are several naturally occurring isoforms of growth hormone produced by two genes, one expressed in the pituitary, human growth hormone-N (“hGH-N” also known as “hGH-1”) and one expressed in the placenta, human growth hormone-variant (“hGH-V” also known as “hGH-2”). The major form of hGH-N is a 22 kDa protein consisting of 191 amino acids. A second form of hGH-N is produced by alternative splicing of the same gene, this results in deletion of a region corresponding to amino acids 32-46 of 22 kDa hGH to produce a 20 kDa protein (20 kDa hGH-N) (U.S. Pat. Nos. 6,399,565 and 6,436,674). Various other splice variants of hGH-N have been described (U.S. Pat. Nos. 4,670,393 and 5,962,411).

The hGH-V gene encodes for a 22 kDa hGH-V isoform which differs from 22 kDa hGH-N by 13 amino acids in various positions throughout the hormone sequence (U.S. Pat No. 4,670,393). The 22 kDa hGH-V is secreted by the placenta and appears in maternal serum at mid-pregnancy. The exact function of this variant is still to be elucidated but it is believed to play a role in the control and development of foetal growth.

### SUMMARY

Growth hormone therapy is used in the treatment of a variety of conditions including small stature. Effects of GH on growth are termed “somatogenic effects.” However, conventional GH therapy is subject to the presence of detrimental side effects, including for example, peripheral edema and fluid retention, lactogenic effects, liver damage and cellular damage. It is clearly advantageous to establish a method of eliminating or at least alleviating these side effects, while maintaining desired growth-promoting effects. Embodiments of this invention include methods of reducing the side effects of GH treatment by the use of a variant of GH that has a different spectrum of activity to the 22 kDa hGH-N, which is currently used for GH therapy. This variant provides the beneficial effects of conventional therapy such as growth promotion and lipolysis but unwanted properties are reduced. Hence, this invention is directed at the use of 20 kDa hGH-V in the treatment of conditions that are currently treated with hGH or have the potential to be treated with hGH. In particular, it is directed at methods of treatment whereby the lactogenic side effects of hGH treatment are reduced.

Embodiments of this invention also include methods of increasing levels of growth hormone in a mammal for prophylactic or therapeutic purposes, comprising administering to a mammal a pharmaceutically effective amount of the GH variant 20 kDa hGH-V or a polypeptide that is substantially identical to 20 kDa hGH-V.

In one embodiment, this variant elicits the growth-promoting ability of GH but has a reduced ability to elicit undesired effects of conventional GH therapy. These undesired effects include, but are not limited to diabetogenic and lactogenic effects, peripheral edema, hepatotoxicity and cell damage.

In another embodiment, 20 kDa hGH-V is produced exogenously and administered to the subject. In view of the size of the variant, it is preferably produced by expression of a gene encoding the variant in a suitable host cell. Such a variant gene can be prepared by site-specific mutagenesis of a GH gene. Suitable host cells containing an expression vector containing such a variant gene can be implanted in the animal to be treated, and induction of expression of the variant gene can lead to increased levels of 20 kDa hGH-V product, which can exert therapeutic effects.

### BRIEF DESCRIPTION OF THE FIGURES

This invention is described with respect to specific embodiments thereof. Additional features of this invention are found in the Figures, in which:

FIG. 1 depicts nucleotide sequences of hGH variants. Dashes indicate section deleted in 20 kDa hGH-V and 20 kDa hGH-N.

FIG. 2 depicts predicted amino acid sequences for 22kDa hGH-V, 22 kDa hGH-N, 20 kDa hGH-V and 20 kDa hGH-N. A dash indicates deleted amino acids.

FIG. 3 depicts competitive binding curves of labeled rbGH to ovine hepatic microsomal membranes using 22kDa hGH-N, 22 kDA hGH-V, 20 kDa hGH-V and 20 kDa hGH-N as unlabelled ligands (assay number 0426).

FIG. 4 depicts competitive binding curves of labelled rbGH to ovine hepatic microsomal membranes using 22 kDa hGH-N and 20 kDa hGH-V as unlabelled ligands (assay number 0440).

FIG. 5 depicts competitive binding curves of labelled rhGH (22 kDa hGH-N) to ovine hepatic microsomal membranes using rbGH, 20 kDa hGH-V, 20 kDa hGH-N and 22 kDa hGH-V as unlabelled ligands (assay number 0434).

FIG. 6 depicts competitive binding curves of labelled rhGH (22 kDa hGH-N) to ovine hepatic microsomal membranes using rbGH and 22 kDa hGH-V as unlabelled ligands (assay number 0435)

FIG. 7 depicts competitive binding curves of labelled ovine prolactin (“oPRL”) to ovine hepatic microsomal membranes using 22 kDa hGH-N, 20 kDa hGH-V and 20 kDa hGH-N as unlabelled ligands (assay number 0439).

FIG. 8 depicts competitive binding curves of labelled rhGH to ovine hepatic microsomal membranes using 22 kDa hGH-N, recombinant bovine growth hormone (“rbGH”) and 20 kDa hGH-V as unlabelled ligands (assay number 0441).

FIG. 9 depicts the cumulative change in body weight in grams over the 7 days of treatment with bovine growth hormone (“bGH”) or hGH variants.

FIGS. 10A and 10B depict total body weight change in grams (FIG. 10A) and daily change in body weight in grams (FIG. 10B) following 7 days of treatment with saline, bGH or hGH variants.

FIG. 11 depicts changes in tibial bone length in rats following 7 days of treatment with saline, bGH or hGH variants.

FIG. 12 depicts changes in nose-anus length of rats following 7 days of treatment with saline, bGH or hGH variants.

FIGS. 13A and 13B depict total food intake by rats (FIG. 13A) and food intake adjusted for body weight (FIG. 13B) following 7 days of treatment with saline, bGH or hGH variants.

FIG. 14 depicts retroperitoneal fat pad weight in rats expressed as a percentage of body weight following 7 days treatment with saline, bGH or hGH variants.

FIG. 15 depicts plasma IGF-I concentrations in rats following 7 days treatment with saline, bGH or hGH variants.

FIGS. 16A, 16B and 16C depict fasting plasma free fatty acids (“FFAs”; FIG. 16A), triglycerides (FIG. 16B) and glycerol (FIG. 16C) following 7 days of treatment of rats with either saline, bGH or hGH variants.

FIG. 17 depicts concentrations of alkaline phosphatase in the plasma in rats treated with bGH or hGH variants.

FIG. 18 depicts fasting plasma globulin concentrations in rats treated with bGH or hGH variants.

FIG. 19 depicts fasting plasma amylase concentrations in rats treated with bGH or hGH variants.

FIG. 20 depicts blood hematocrit in rats following 7 days of treatment with either saline, bGH or hGH variants.

FIG. 21 depicts cumulative body weight gain in rats following 7 days of treatment with either saline, bGH or hGH variants. Data are mean±SEM (n=6 per group).

### DETAILED DESCRIPTION

Definitions

‘Transformation’ means introducing DNA into an organism so that the DNA is replicable, either as an extrachromosomal element or by chromosomal integration. Methods of transformation may be the method of, for example, Graham and van der Eb, Virology 52: 456-457 (1973) or by other suitable methods for introducing DNA into cells such as by nuclear injection or by protoplast fusion. For cells which contain substantial cell wall constructions, such as prokaryote cells, transfection can be by calcium treatment as described by Cohen et al, Proc. Natl. Acad. Sci. (USA), 69: 2110 (1972). Additional methods are well known in the art and can be found in Sambrook and Russell, Molecular Cloning Third Edition, Cold Springs Harbor Laboratory Press, New York (2001).

‘Transfection’ means the introduction of DNA into a host cell whether or not any coding sequences are ultimately expressed. Cells do not naturally take up DNA thus, a variety of technical methods are utilised to facilitate gene transfer. Methods of transfection will be known to those skilled in the art and include for example, CaPO₄ and electroporation. Additional methods are well known in the art and can be found in Sambrook and Russell, Id.

‘Conservative amino acid substitutions’ mean amino acid substitutions or deletions that do not substantially affect the character of the variant polypeptide relative to the starting peptide. For example substitutions can be made within the following four groups: 1) positively charged residues e.g. Arg, Lys, His. 2) negatively charged residues e.g. Asn, Asp, Glu, Gln. 3) bulky aliphatic residues e.g. Ile, Leu, Val. 4) bulky aromatic residues e.g. Phe, Tyr, Trp. For further identification of conservative substitutions see, for example, Livingstone and Barton, Comput. App. Biosci. 9(6) 745-756, 1993.

‘Substantially identical’ refers to a polypeptide that has a sequence wherein one or two amino acid insertions, substitutions or deletions have been made or where conservative amino acid substitutions have been made such that the polypeptide thus formed does not materially differ in character and activity from 20 kDa hGH-V and has at least 95% homology to the nucleotide sequence SEQ ID NO: 3. “Substantially identical” also refers to an oligonucleotide sequence that differs from a first oligonucleotide sequence by “silent” differences based on redundancy of the genetic code (e.g., in which the difference does not result in any change in amino acid). Alternatively, “substantially identical” sequences are oligonucleotide sequences that can hybridise to a complement of a first oligonucleotide sequence under stringent conditions, for example, 0.1 SSC, 60° C. for 1 hour. It can be appreciated that other conditions of stringency can be selected and still retain high-fidelity hybridization.

‘hGH-N’ means pituitary human growth hormone.

‘hGH-V’ means placental human growth hormone.

‘PRL’ means prolactin.

‘PRLR’ means prolactin receptor.

‘20 kDa hGH-V’ means a polypeptide with the amino acid sequence of SEQ ID NO:7, or the nucleotide sequence of SEQ ID NO:3, or a polypeptide that is substantially identical to a polypeptide with the amino acid sequence of SEQ ID NO:7, or an oligonucleotide substantially identical to the nucleotide sequence of SEQ ID NO:3.

‘20 kDa hGH-N’ means a polypeptide with the amino acid sequence of SEQ ID NO:8, or the nucleotide sequence of SEQ ID NO:4, or a polypeptide that is substantially identical to a polypeptide with the amino acid sequence of SEQ ID NO:8, or an oligonucleotide substantially identical to the nucleotide sequence of SEQ ID NO:4.

‘22 kDa hGH-V’ means a polypeptide with the amino acid sequence of SEQ ID NO:5, the nucleotide sequence of SEQ ID NO:1, or a polypeptide that is substantially identical to a polypeptide with the amino acid sequence of SEQ ID NO:5, or an oligonucleotide substantially identical to the nucleotide sequence of SEQ ID NO:1.

‘22 kDa hGH-N’ means a polypeptide with the amino acid sequence of SEQ ID NO:6, or the nucleotide sequence of SEQ ID NO:2, or a polypeptide that is substantially identical to a polypeptide with the amino acid sequence of SEQ ID NO:6, or an oligonucleotide substantially identical to the nucleotide sequence of SEQ ID NO:2.

‘Somatogenic effects’ includes growth-promoting, body-weight increasing and osteo-anabolic actions.

‘Lactogenic effects’ includes effects of exogenous growth hormone that are associated with PRLR signalling. Those effects include but not limited to: mammary gland development, changes in osmotic balance and cell proliferation.

‘Metabolic effects’ include, but are not limited to stimulation of lipolysis, stimulation of secretion of IGF-1, and diabetogenic effects.

Growth Hormone Therapy

There is an unmet need for methods and medicaments that have the beneficial growth promoting effects of GH but has reduced side effects.

Therefore, in certain aspects, this invention provides a method of treating a condition in a mammal, comprising administering to the mammal a pharmaceutically effective amount of 20 kDa hGH-V or a polypeptide that is substantially identical to 20 kDa hGH-V.

In other aspects, the method includes treating adult-onset growth hormone deficiency, childhood-onset growth hormone deficiency, cystic fibrosis, osteoporosis, skeletal dysplasia, chronic kidney failure, depression, memory loss, catabolic states, anorexia and hypertension.

In other aspects, the invention comprises a pharmaceutical composition comprising a 20 kDa hGH-V and a pharmaceutically acceptable excipient.

In still further aspects, this invention includes pharmaceutical composition comprising a 20 kDa hGH-V, a pharmaceutically acceptable excipient and a binder.

In further aspects, this invention includes a pharmaceutical composition comprising a 20 kDa hGH-V, a pharmaceutically acceptable excipient and a capsule.

Further aspects of this invention include a method for treating a patient in need of growth hormone therapy, comprising administering to a patient a 20 kDa hGH-V.

In certain of these aspects, a method includes administering an expression vector capable of producing 20 kDa hGH-V.

In further aspects, the expression vector is in a host cell.

In yet other aspects, theexpression vector is in a cell of the patient.

In other aspects, the invention includes administering to a mammal in need of growth hormone therapy, a composition comprising 20 kDa hGH-V.

In still other aspects, the invention includes administering to said mammal, a cell having a replicable vector therein capable of producing 20 kDa hGH-V.

Side Effects of GH

Undesirable side effects of conventional GH therapy using 22 kDa pituitary GH include one or more of: oedema, fluid retention, hypertension, benign intracranial hypertension; glucose intolerance and/or diabetes; gynaecomastia; musculoskeletal effects such as arthralgia, paresthesias and carpal tunnel syndrome or myalgia.

Oedema is defined as an accumulation of an excessive amount of watery fluid in cells, tissues or serous cavities (such as the abdomen). Symptoms of extracellular edema include puffiness of the face around the eyes, or in the feet, anlles and legs. GH induced salt and water retention can cause peripheral edema or benign intracranial hypertension.

Benign intracranial hypertension is characterized by increased cerebrospinal fluid pressure in the absence of a space-occupying lesion. It can present with headache, visual loss, nausea, vomiting and papilloedema.

There is increasing concern over diabetogenic effects of GH therapy especially during childhood. GH therapy has been shown to cause glucose intolerance and reduce insulin sensitivity. An increased incidence has been established between GH therapy and type 2 diabetes mellitus in some groups of children and adolescents (Cutfield 2000). Hyperglycaemia has also been observed in adults undergoing GH treatment.

Arthralgia is pain in one or more joints. Paresthesia is a term that refers to an abnormal burning or prickling sensation which is generally felt in the hands, arms, legs, or feet, but can occur in any part of the body. Carpal tunnel syndrome occurs when tendons or ligaments in the wrist become enlarged, often from inflammation. The narrowed tunnel of bones and ligaments in the wrist pinches the nerves that reach the fingers and the muscles at the base of the thumb. Symptoms range from a burning, tingling numbness in the fingers, especially the thumb and the index and middle fingers, to difficulty gripping or making a fist, to dropping things.

Myalgia is pain or discomfort moving any muscle(s).

There has been some concern about the possibility of “cancer growth promotion” with growth hormone therapy, based upon a few cases of leukaemia reported in children treated with growth hormone therapy (Stahnke & Zeisel, 1989; Sartorio et al, 1989; Watanabe et al, 1988).

Conditions Treated Using GH

GH therapy is used to treat a varied range of conditions. At present, the prophylactic or therapeutic efficacy of GH has been established or indicated with regard to conditions that include, but are not limited to: adult-onset growth hormone deficiency (caused mainly by pituitary adenoma, surgery, or radiation therapy); childhood-onset growth hormone deficiency caused by: (a) congenital conditions (anatomical abnormalities or genetic factors), (b) acquired conditions (CNS tumours, cranial irradiation, infiltrative diseases, trauma, hypoxic insult) or (c) idiopathic causes; cystic fibrosis, osteoporosis, chronic kidney failure, depression, memory loss, catabolic states, anorexia, hypertension.

GH therapy is approved for use in growth hormone deficiency in children, Prader-Willi syndrome, growth hormone deficiency in adults, Turner syndrome, chronic renal insufficiency and AIDS-associated wasting. Growth hormone is also useful in the treatment of several other conditions. These conditions include constitutional delay of growth, cystic fibrosis, osteoporosis, depression, memory loss, catabolic states and hypertension.

GH Deficiency

Diagnosis of growth hormone deficiency requires growth hormone stimulation testing. Tests used include the insulin hypoglycemia test or insulin tolerance test (ITT), L-dopa stimulation test, arginine infusion test and arginine/GHRH test. Peak growth hormone secretion levels in adults of less than 3-5 ng/mL are indicative of GHD. In children values below 10 ng/mL are considered inadequate. Growth hormone deficiency is treated with recombinant human growth hormone which is usually given via a subcutaneous injection on a daily basis.

There are several causes of GHD in children and most can be related to a problem in the hypothalamus or the pituitary. In certain rare cases, a defect in the body's utilization of growth hormone occurs. In most children with growth hormone deficiency, the defect lies in the hypothalamus. When other pituitary hormones are also not being secreted normally, the child is said to have hypopituitarism. In congenital hypopituitarism, abnormal formation of the pituitary or hypothalamus occurs during fetal development. Acquired hypopituitarism results from damage to the pituitary or hypothalamus that occurs during or following birth. It can be caused by a severe head injury, brain damage due to disease, radiation therapy, or a tumour.

The worldwide incidence of GHD in children has been estimated to be at least 1 in 10,000 live births and some individual countries have reported an incidence as high as 1 in 4,000 live births. A growth hormone deficient child usually shows a growth pattern of less than 2 inches a year. In many cases the child will grow normally until the age of 2 or 3 and then begin to show signs of delayed growth. Testing for growth hormone deficiency will occur when other possibilities of short stature have been ruled out. A weekly dose of up to 0.30 mg/kg of body weight divided into daily subcutaneous injections is recommended for GHD children.

In adults, deficiency of growth hormone can develop in the following situations; presence of a large pituitary tumour, after surgery or radiation therapy of pituitary tumour or other brain tumours, secondary to hypothalamic disorders and the continuation of childhood growth hormone deficiency into adulthood. The clinical features of adult GHD include; fatigue, muscle weakness, reduced exercise capacity, weight gain, increase in body fat and decrease in muscle mass, increase in LDL cholesterol and triglycerides and decrease in HDL cholesterol, increased risk for heart attack, heart failure and stroke, decrease in bone mass, anxiety and depression, especially lack of sense of well-being, social isolation and reduced energy. In the United States, an estimated total of 35,000 adults have GHD and approximately 6,000 new cases of GHD occur each year. For the average 70 kg man, the recommended dosage at the start of therapy is approximately 0.3 mg given as a daily subcutaneous injection. The dose can be increased, on the basis of individual requirements, to a maximum of 1.75 mg daily in patients younger than 35 years of age and to a maximum of 0.875 mg daily in patients older than 35 years. Lower doses may be needed to minimize the occurrence of adverse events, especially in older or overweight patients.

Prader-Willi Syndrome

Prader-Willi syndrome is a disorder of chromosome 15 characterised by hypotonia, hypogonadism, hyperphagia, cognitive impairment and difficult behaviour; the major medical concern being morbid obesity. Growth hormone is typically deficient, causing short stature, lack of pubertal growth spurt, and a high body fat ratio, even in those with normal weight. The need for GH therapy should be assessed in both children and adults. In children, if growth rate falls or height is below the third percentile, GH treatment should be considered. Growth hormone replacement helps to normalize the height and increases lean body mass; these both help with weight management. The usual weekly dose is 0.24 mg/kg of body weight; this is divided into 6 or 7 smaller doses over the course of the week.

Turner Syndrome

Turner syndrome occurs in approximately 1 in 2,500 live-born girls. It is due to abnormalities or absence of an X chromosome and is frequently associated with short stature, which can be ameliorated by GH treatment. Other features of Turner syndrome can include shortness of the neck and at times, webbing of the neck, cubitus valgus, shortness of fourth and fifth metacarpals and metatarsals, a shield shaped chest and primary hypogonadism. Growth in height is variable in patients with Turner syndrome so the decision whether to treat with GH and the timing of such treatment is made on an individual basis. Often, treatment is initiated when a patient's height declines below the 5^{th} percentile or when the standard deviation score decreases to less than 2 standard deviations below the mean. Treatment is often initiated with GH doses slightly higher than those used in treating GHD; a common starting dosage is 0.375 mg/kg per week divided into daily doses.

Chronic Renal Insufficiency

Chronic renal insufficiency (CRI) affects about 3,000 children in the United States. It manifests through a gradual and progressive loss of the ability of the kidneys to excrete wastes, concentrate urine, and conserve electrolytes. Approximately a third of children with chronic renal disease have abnormal growth partly because renal diseases disturb the metabolism of growth hormone. The corticosteroid hormones which are often used to treat the kidney disease can also retard growth. Kidney transplants can help a child start growing normally again, but most children do not make up the growth lost prior to transplantation. The age that the renal disease starts has more impact on growth retardation than the reduction in renal function (i.e. the younger the child when the disease starts, the more retarded is his or her growth). GH treatment can be given at a dosage of 0.35 mg/kg per week given six or seven times weekly.

HIV Wasting Syndrome

A common problem among HIV-infected people is the HIV wasting syndrome, defined as unintended and progressive weight loss often accompanied by weakness, fever, nutritional deficiencies and diarrhoea. The syndrome, also known as cachexia, can diminish the quality of life, exacerbate illness and increase the risk of death for people with HIV. The body consumes muscle and organ tissue for energy instead of primarily relying on the body's stored fat.

Wasting can occur as a result of HIV infection itself but also is commonly associated with HIV-related opportunistic infections and cancers. HIV wasting syndrome is diagnosed in HIV-infected people who have unintentionally lost more than 10 percent of their body weight. Most patients with advanced HIV disease and AIDS eventually experience some degree of wasting. Estimates of the prevalence of AIDS wasting range from 4-30% of HIV infected individuals. GH treatment is in the order of 0.1 mg/kg daily.

Constitutional Delay of Growth

Constitutional delay of growth is characterized by normal prenatal growth followed by growth deceleration during infancy and childhood, and is reflected in declining height percentiles at this time. Between 3 years of age and late childhood, growth proceeds at a normal velocity. A period of pronounced growth deceleration can be observed immediately preceding the onset of puberty. Children with constitutional delay have later timing of puberty. At times, the combination of short stature accompanied and exaggerated by constitutional delay of growth and development in adolescents can cause sufficient psychosocial adolescent stress to warrant treatment with GH administered in the same manner and dosage as that used for treating GHD.

Cystic Fibrosis

Cystic Fibrosis (CF) is the most common lethal genetic disorder in America. An estimated 1000 individuals are born with Cystic Fibrosis each year in the United States. Cystic fibrosis causes dysfunction of the exocrine glands with increased viscosity of mucus secretions, which leads to pulmonary disease, exocrine pancreatic insufficiency, and intestinal obstruction. Early diagnosis and treatment has significantly decreased mortality in children with CF. However, malnutrition and poor growth continue to be a significant problem. Poor weight gain, weight loss, and inadequate nutrition result from reduced energy intake, increased energy loss, and increased energy expenditure. It has been reported that 28% of persons with CF are below the 10th percentile for height and 34% are below the 10th percentile for weight. Studies have shown that GH therapy improves height velocity, weight velocity, lean body mass (LBM) and pulmonary function in patients with cystic fibrosis.

Osteoporosis

Osteoporosis is a disease characterized by low bone mass and structural deterioration of bone tissue, leading to bone fragility and an increased susceptibility to fractures, especially of the hip, spine and wrist. Osteoporosis is responsible for more than 1.5 million hip fractures annually world wide. Most fractures occur in postmenopausal women, however, approximately one third of all osteoporotic fractures occur in men. Treatment of osteoporosis with GH might be beneficial due to the increased bone metabolism and improved bone geometry which occurs with GH. The GH/IGF-I system is dysregulated in patients with post-menopausal osteoporosis. This is shown by reduced systemic IGF and IGFBP-3-levels in osteoporosis suggesting a decrease of endogenous GH-secretion or a dysregulation of the GH receptor system which is beyond the normal ageing process of the GH/IGF system, the “somatopause”. Studies have shown that GH treatments can improve bone mineral density in men with idiopathic osteoporosis.

Skeletal Dysplasias

Skeletal dysplasias associated with short stature such as achondroplasia can be treated with GH. Achondroplasia is a genetic disorder, affecting the fibroblast growth factor receptor type III gene, which is evident at birth. It affects about one in every 20,000 births and it occurs in all races and in both sexes. During fetal development and childhood, cartilage normally develops into bone, except in a few places, such as the nose and the ears. In individuals with achondroplasia the rate, at which cartilage cells in the growth plates of the long bones turn into bone is slow, leading to short bones and reduced height.

Achondroplasia is characterized by short stature, short limbs, proximal extremity (upper arm and thigh), head appears disproportionately large for body, skeletal (limb) abnormalities, abnormal hand appearance (trident hand) with persistent space between the long and ring fingers, marked kyphosis and lordosis (spine curvatures), waddling gait, bowed legs, prominent (conspicuous) forehead (frontal bossing), hypotonia and polyhydramnios (present when affected infant is born). GH has been approved to treat achondroplasia in some countries such as Japan and South Africa but does not yet have FDA approval.

Catabolic Protein Wasting States

Catabolic states are characterised by protein wasting. Growth hormone treatment can be used to prevent excessive protein loss. Such catabolic states can exist in patients after long-term fasting, anorexia, chronic disease, prolonged immobilisation, trauma, burns and extensive surgery. GH and insulin-like growth factor I (IGF-I) play a physiological role in the regulation of protein metabolism in catabolic conditions. During such conditions the GH axis is frequently disturbed.

Lipodystrophy

GH can also be beneficial for the treatment of lipodystrophy, particularly for AIDS associated lipodystrophy. Lipodystrophy is a generic term that simply means a disturbance of fat metabolism. HIV-related lipodystrophy generally consists of fat accumulation in the following areas: subcutaneous tissues of the lower trunk (abdominal region), abdominal viscera (visceral obesity), axillary pads (bilateral, symmetric lipomatosis) and dorsocervical region (-so-called buffalo hump) and loss of fat from the subcutaneous tissues of the following areas: lower extremities, upper extremities, buttocks and face (maxillary, nasolabial, and temporal regions). This syndrome of HIV-related lipodystrophy appears to be quite distinct from the wasting syndrome of protein-energy malnutrition. There is no universally agreed-on case definition of lipodystrophy in the HIV-infected patient so the diagnosis depends, to a certain extent, on a physician's clinical judgment. Skin-fold measurements or hip-to-waist ratios are neither very accurate nor reproducible. Single-slice CT scan at the level of the fourth lumbar vertebra is the most reproducible test, but it is also the most expensive.

Intrauterine Growth Retardation and Children of Small Gestational Age

GH treatment can be beneficial in children with inter uterine growth retardation (“IUGR”) or infants who are small for gestational age (a condition also termed Russell-Silver syndrome; “SGA Children”). One definition of inter uterine growth retardation is a weight below the 10^{th} percentile for gestational age or a birth weight 2 standard deviations below the mean for gestational age. Studies have shown that those children who don't show catch-up growth can benefit from GH treatment.

Osteogenesis Imperfecta

Osteogensis imperfecta (OI) is caused by mutations in the gene for type I collagen. It is associated with bone de-mineralization and, in many instances, with retarded bone growth. OI is characterized by bones that break easily often from little or no apparent cause. While the number of people affected with OI in the United States is unknown, the best estimate suggests a minimum of 20,000 and possibly as many as 50,000. It is often, though not always, possible to diagnose OI based solely on clinical features. Clinical geneticists can also perform biochemical (collagen) or molecular (DNA) tests that can help confirm a diagnosis of OI in some situations. In some cases osteogenesis imperfecta can be effectively treated with GH. In particular, patients can experience improved bone mineralization and improved growth.

Inflammatory Bowel Disease

GH can be used for the treatment of inflammatory bowel disease, Crohn's disease and short bowel syndrome. Inflammatory bowel disease (IBD) is a group of disorders that cause inflammation or ulceration of the digestive tract. Depending on the type of IBD, any part of the digestive tract from the mouth to the anus can be affected. The small and large intestines, the rectum, and the anus are affected most often. Ulcerative colitis and Croin's disease are the most common types of inflammatory bowel disease. The cause of IBD is not known however, it is believed to develop in people who have a genetic tendency. In these individuals, the immune system can overreact to normal intestinal bacteria, causing inflammation. The main symptoms are abdominal pain, rectal bleeding, and diarrhoea or constipation. Fever and loss of appetite also can occur. Short bowel syndrome is characterized by massive loss of intestine, with impaired net absorptive capacity of the remaining gut. Patients without colon often face problems with sodium/fluid balance and often require nutritional support due to malnutrition of several nutrients.

Glucocorticord Induced Growth Retardation

GH treatment can be considered in extremely short persons with growth retardation attributable to glucocorticoid treatment. The glucocorticoid regimen should be reduced to the minimal dose needed to achieve a satisfactory clinical effect before initiation of GH therapy in such patients.

Other Conditions

Other conditions that can benefit from GH treatment include depression, memory loss, obesity, hypertension, infertility and the like. However, it can be readily appreciated that any condition that can benefit from GH therapy can be treated advantageously using methods and medicaments of this invention.

Therapy Using Pituitary GH

Current conventional GH therapy uses 22 kDa pituitary GH. The 22 kDa and 20 kDa versions of pituitary GH are thought to have equivalent somatogenic activity. 20 kDa hGH-N was equivalent to 22 kDa hGH-N in a growth-promoting assay in spontaneous dwarf rats (Ishikawa 2000, Ishikawa 2001), the osteo-anabolic effect of 20 kDa was equipotent to that of 22 kDa (Wang 1999), cell proliferation of full length hGH-R-expressing cells was stimulated equipotently (Wada 1998) and 20 kDa hGH-N was shown to be a full agonist in hypophysectomized rats (Uchida 1997). A 20 kDa hGH-N has also been shown to produce inhibition of LPL activity in adipose tissue and to stimulate lipolysis in adipocytes in a manner similar to 22 kDa hGH-N (Takahashi 2002). The lipolytic activity of 20 kDa hGH-N may be higher than 22 kDa hGH-N in the presence of growth hormone binding protein (“GHBP”) (Asada 2000).

However, 22 kDa hGH-N is known to induce insulin resistance. Studies indicate that the diabetogenicity of 20 kDa hGH-N is much weaker than 22 kDa hGH-N. 20 kDa hGH-N was shown to much less potent than 22 kDa hGH-N at inducing insulin resistance in euglycaemic clamp studies (Takahashi 2001) and in studies using GH deficient dwarf rats (Ishikawa 2001).

20 kDa hGH-N is a much weaker agonist for the prolactin receptor than 22 kDa hGH-N and hence lacks some of the lactogenic properties of 22 kDa hGH-N (Tsunekawa 1999) as the lactogenic effects of GH are believed to be mediated by the prolactin receptor. It has been suggested that administration of 20 kDa hGH-N may alleviate hPRLR-mediated side-effects such as breast cancer (Tsunekawa 1999). 20 kDa hGH-N also has different antidiuretic effects to 22 kDa hGH-N. Administration of 22 kDa hGH-N suppressed urine excretion in intact rats whereas 20 kDa hGH-N showed no significant effect (Satozawa 2000), this is significant as fluid retention can cause oedema. 20 kDa hGH-N is thought to lack part of the PRLR binding region of 22 kDa hGH-N. Tables 1 and 2 below show the oligonucleotide and amino acid sequences of growth hormone variants useful in the methods of this invention. In Table 1, sequence identification numbers below are for oligonucleotides: 22 kDa hGH-V (SEQ ID NO:1), 22 kDa hGH-N (SEQ ID NO:2), 20 kDa hGH-V (SEQ ID NO:3) and 20 kDa hGH-N (SEQ ID NO:4).

TABLE 1
Translated nucleotide sequence of hGH variants.

| | |
|---|---|
| SEQ ID NO:1 | ATG GCT GCA GGC TCC CGG ACG TCC CTG CTC CTG GCT TTT GGC CTG CTC TCG |
| SEQ ID NO:2 | ACA |
| SEQ ID NO:3 | GCA |
| SEQ ID NO:4 | ACA |
| | CTG TCC TGG CTT CAA GAG GGC AGT GCC TTC CCA ACC ATT CCC TTA TCC AGG |
| | CCC |
| | TCC |
| | CCC |
| | CTT TTT GAC AAC GCT ATG CTC CGC GCC CGT CGC CTG TAC CAG CTG GCA TAT |
| | CAT CGT CAC GCC TTT |
| | CGT CGC TAC GCA TAT |
| | CAT CGT CAC GCC TTT |
| | GAC ACC TAT CAG GAG TTT GAA GAA GCC TAT ATC CTG AAG GAG CAG AAG TAT |
| | TAC CTA GAA |
| | TAT --- --- --- --- --- --- --- --- --- --- --- |
| | TAC --- --- --- --- --- --- --- --- --- --- --- |
| | TCA TTC CTG CAG AAC CCC CAG ACC TCC CTC TGC TTC TCA GAG TCT ATT CCA |
| | TGT CCG |
| | --- --- --- --- TGC CCA |
| | --- --- --- --- TGT CCG |
| | ACA CCT TCC AAC AGG GTG AAA ACG CAG CAG AAA TCT AAC CTA GAG CTG CTC |
| | CCC GAG GAA ACA CAA TCC |
| | CCT GTG AAA ACG CAG TCT |
| | CCC GAG GAA ACA CAA TCC |
| | CGC ATC TCC CTG CTG CTC ACT CAG TCA TGG CTG GAG CCC GTG CAG CTC CTC |
| | TCG TCT |
| | TCA CTC |
| | TCG TCT |
| | AGG AGC GTC TTC GCC AAC AGC CTG GTG TAT GGC GCC TCG GAC AGC AAC GTC |
| | AGT TAC TCT |
| | AGC TAT TCG |
| | AGT TAC TCT |
| | TAT CGC CAC CTG AAG GAC CTA GAG GAA GGC ATC CAA ACG CTG ATG TGG AGG |
| | GAC CTC CTA GGG |
| | CGC CAC CTG TGG |
| | GAC CTC CTA GGG |
| | CTG GAA GAT GGC AGC CCC CGG ACT GGG CAG ATC TTC AAT CAG TCC TAC AGC |
| | AAG ACC |
| | AAT TCC |
| | AAG ACC |
| | AAG TTT GAC ACA AAA TCG CAC AAC GAT GAC GCA CTG CTC AAG AAC TAC GGG |
| | TTC AAC TCA CTA |
| | TTT AAA TCG CTG |
| | TTC AAC TCA CTA |
| | CTG CTC TAC TGC TTC AGG AAG GAC ATG GAC AAG GTC GAG ACA TTC CTG CGC |
| | ATC GTG CAG TGC CGC TCT GTG GAG GGC AGC TGT GGC TTC TAG |

Dashes indicate section deleted in 20 kDa hGH-V and 20 kDa hGH-N.
Underlined section indicates signal sequence.

TABLE 2

Predicted amino acid sequences for 22 kDa hGH-V,
22 kDa hGH-N, 20 kDa hGH-V, 20 kDa hGH-N.

| | |
|---|---|
| | 20 |
| SEQ ID NO:5 | Phe Pro Thr Ile Pro Leu Ser Arg Leu Phe Asp Asn Ala Met Leu Arg Ala Arg Arg Leu |
| SEQ ID NO:6 | Ser His |
| SEQ ID NO:7 | Met Arg |
| SEQ ID NO:8 | Ser His |
| | 40 |
| | Tyr Gln Leu Ala Tyr Asp Thr Tyr Gln Glu Phe Glu Glu Ala Tyr Ile Leu Lys Glu Gln |
| | His Phe Pro |
| | Tyr Tyr --- --- --- --- --- --- --- --- --- |
| | His Phe --- --- --- --- --- --- --- --- --- |
| | 60 |
| | Lys Tyr Ser Phe Leu Gln Asn Pro Gln Thr Ser Leu Cys Phe Ser Glu Ser Ile Pro Thr |
| | --- --- --- --- --- --- |
| | --- --- --- --- --- --- |
| | 80 |
| | Pro Ser Asn Arg Val Lys Thr Gln Gln Lys Ser Asn Leu Glu Leu Leu Arg Ile Ser Leu |
| | Glu Glu |
| | Val Lys |
| | Glu Glu |
| | 100 |
| | Leu Leu Ile Gln Ser Trp Leu Glu Pro Val Gln Leu Leu Arg Ser Val Phe Ala Asn Ser |
| | Phe |
| | Leu |
| | Phe |
| | 120 |
| | Leu Val Tyr Gly Ala Ser Asp Ser Asn Val Tyr Arg His Leu Lys Asp Leu Glu Glu Gly |
| | Asp Leu |
| | Arg His |
| | Asp Leu |
| | 140 |
| | Ile Gln Thr Leu Met Trp Arg Leu Glu Asp Gly Ser Pro Arg Thr Gly Gln Ile Phe Asn |
| | Gly Lys |
| | Trp Asn |
| | Gly Lys |
| | 160 |
| | Gln Ser Tyr Ser Lys Phe Asp Thr Lys Ser His Asn Asp Asp Ala Leu Leu Lys Asn Tyr |
| | Thr Asn |
| | Ser Lys |
| | Thr Asn |
| | 180 |
| | Gly Leu Leu Tyr Cys Phe Arg Lys Asp Met Asp Lys Val Glu Thr Phe Leu Arg Ile Val |
| | 191 |
| | Gln Cys Arg Ser Val Glu Gly Ser Cys Gly Phe |

A dash indicates deleted amino acids

Table 2 shows amino acid sequences for 22 kDa hGH-V (SEQ ID NO:5), 22 kDa hGH-N (SEQ ID NO:6), 20 kDa hGH-V (SEQ ID NO:7) and 20 kDa hGH-N (SEQ ID NO:8).
Therapy Using Placental GH Variants

22 kDa hGH-V has been shown to have similar somatogenic but reduced lactogenic activity compared to the hGH-N isoform (Igout 1995). 22 kDa hGH-V binds to somatogen receptors (Ray 1990) and stimulated growth in hypophysectomized rats (MacLeod 1991). 22 kDa hGH-V binds to both somatogen and lactogen receptors but the ratio of its somatogen to lactogen receptor-binding affinities is higher than that of 22 kDa hGH-N. This ratio differed by 7-8 fold in experiments using rat liver lactogen receptors (Ray 1990) and by 30 fold using Nb2 cell lactogen receptors (MacLeod 1991). The lipolytic and insulin-like activities of 22 kDa hGH-N and 22 kDa hGH-V have been shown to be similar in rat adipose tissue (Goodman 1991).

A second splice variant of the GH-V gene retains intron D in the mRNA to give a 26 kDa hGH-V isoform (hGH-V2) (Cooke 1988). Recently two new transcripts of the hGH-V gene have been described (Boguszewski 1998). hGH-V3 is generated by alternative splicing near the end of the fourth exon to predict a 24 kDa protein (219 amino acids) wherein the carboxy-terminal residues show complete sequence divergence from hGH-V. The second transcript to be described uses a similar alternative splice site within exon 3, to that seen for hGH-N, to predict a 20 kDa isoform of hGH-V (GenBank accession number: AF006060).

The transcript for 20 kDa hGH-V had not previously been detected and it was thought that the hGH-V gene did not use this splice site (Cooke 1998, Estes 1992) however, Boguszewski et al detected the transcript of this isoform in two of four full term placentas and in one abnormal placenta (Boguszewski 1998). The difference in expression of this transcript, as the transcript was not found in all placentas, may partly explain the lack of previous detection. While the transcript has been detected the encoded protein has not been isolated and hence, the biological activity was unknown.

It does not follow that the knowledge of the existence of the above transcript means that it can be synthesized or that the biological activity can be predicted. For example, 20 kDa hGH-N proved difficult to obtain. 20 kDa hGH-N can be purified from the pituitary in small amounts but complete separation from 22 kDa hGH-N is difficult due to similarity in physiochemical properties between the two hormones. Methionyl 20 kDa hGH-N has been expressed in E. coli. However, the additional methionine residue at the N-terminal may affect biological activity and it is believed that the protein may also be incorrectly folded as has been the case for methionyl 22 kDa hGH-N (Hsiung 1988). Methionyl 20 kDa hGH-N was expressed at only one-twentieth of the levels of 22 kDa hGH-N and 20 kDa hGH-N produced in COS-7 cells was reported to be secreted at one-thirtieth the rate as compared to that of 22 kDa hGH-N (Rincón-Limas 1993) hence, the development of an efficient synthesis by Uchida et al was not straightforward (Uchida 1997).

Early work on 20 kDa hGH-N isolated from the pituitary and with a non-authentic recombinant product gave quite different results to studies on an ‘authentic’ version (Uchida 1997). Early studies on 20 kDa hGH-N purified from the pituitary indicated that the lipolysis activity of 20 kDa hGH-N was much weaker than 22 kDa hGH-N (Frigeri 1979, Juárez-Aguilar 1995). This did not agree with results obtained using recombinant 20 kDa hGH-N with an authentic sequence (Asada 2000, Takahashi 2002). Methionyl 20 kDa hGH-N has been shown to induce glucose intolerance (Kostyo 1985) and impair insulin sensitivity (Ader 1987) however, more recent studies on 20 kDa hGH-N indicate that the diabetogenicity of 20 kDa hGH-N is much weaker than 22 kDa hGH-N (Takahashi 2001, Ishikawa 2001). Such discrepancies in the literature describing the biological properties of 20 kDa hGH-N produced by different methods indicate that it is obviously not straightforward to predict and demonstrate said properties.

Novel features of the present invention include the surprising finding that 20 kDa hGH-V does not bind to PRLR and therefore does not display any of the lactogenic side-effects associated with GH-N replacement therapy. The ligand binding studies described in Example 1 show that 20 kDa hGH-V has a profile of a pure somatogen. The somatogenic efficacy of 20 kDa hGH-V observed in the binding studies was confirmed in the in vivo studies carried out by the inventors (Example 2).

Administration of the 20 kDa hGH-V variant maintains the somatogenic effects as well as lipolytic effects of 22 kDa hGH-N, but removes the lactogenic effects of conventional therapy using 22 kDa GH-N. Human GH is known to bind an activate both hGH receptor (HGHR) and human PRL receptor. 22 kDa hGH-N action via the PRLR receptor has been associated with fluid retention (Satozawa 2000; Prod Info Humatrope®, 2003; Prod Info Norditropin®, 2001; Prod Info Serostim®, 2003); gynaecomastia (Prod Info Humatrope®, 2003; Prod Info Nutropin®, 2003; Prod Info Nurtopin AQ®, 2003; Prod Info Genotropin®, 2003 and proliferation of tumor cells and tumor growth (Bole-Feysot, 1998).

Prolactin receptor signalling has been shown to reduce renal sodium and potassium excretion (Richardson et al., Br J Pharmacol 47:623P-624P 1973), stimulate Na⁺-K⁺ adenosine triphosphatase (ATPase) (Pippard et al. 1986 J Endocrinology 108:95-99), decrease sodium in sweat (Robertson et al. 1986 Endocrinology 119:2439-2444) and increase water and salt absorption in all regions of the intestine (Mainoya et al. 1974 Endocrinology 63: 311-317). The resulting increase in sodium levels in plasma is associated with water retention. An increase in extracellular fluid volume may result in oedema. Additionally, increased fluid volume in plasma may lead to an increase in blood pressure. In elderly patients with distorted water and salt homeostasis a higher mortality rate has been noted. (Kokko, Juha P., Water and Sodium Regulation in Health and Disease. In: Nature Encyclopedia of Life Sciences. London: Nature Publishing Group; August 1999).

Prolactin receptor signalling has been associated with development of mammary gland. 22 kDa GH-N has been associated, among others, with the development in the male of breasts resembling those of the sexually mature female (Harman S M. 2004 J Gerontol A Biol Sci Med Sci. 59(7):B652-8).

Additionally, prolactin's actions have been associated with different forms of cancer: increase in colorectal tumor agressivity (Bhatavdekar et al. 1994 J Surg Oncol 55:246-249), proliferation of several lines of human breast cancer (Kiss et al. 1987 J Natl Cancer Inst 78:993-998), proliferation of human BPH epithelial cells (Syms et al. 1985 Prostate 6:145-153). 22 kDa GH has been shown to participate in the development of prostate cancer (Weiss-Messer et al. 2004 Mol Cell Endocrinol. 220(1-2): 109-23).

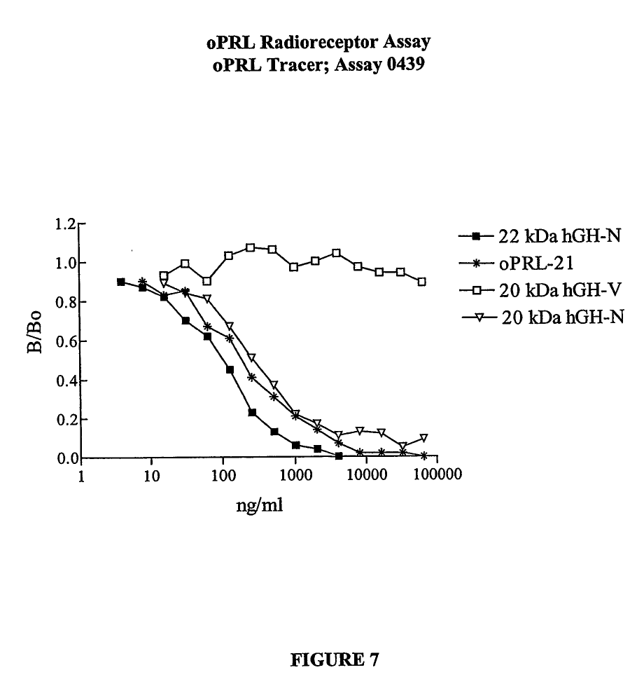

The Inventors have found that 20 kDa hGH-V, 22I:Da hGH-V and 20 kDa hGH-N are more beneficial as GH replacement therapies than the conventional 22 kDa hGH-N or hGH. The Inventor's discovery that 20 kDa hGH-V, 22 kDa hGH-V and 20 kDa hGH-N have (1) desirable somatogenic effects, (2) less binding affinity to prolactin receptors and (3) less undesirable side effects (e.g., lactogenic effects), these variants can provide practitioners with desirable alternatives to conventional therapy. We have unexpectedly found that 20 kDa hGH-V has even less undesirable side effects than either 20 kDa hGH-N or 22 kDa hGH-V. Although 20 kDa hGH-V exerts lesser side effects than 20 kDa hGH-N or 22 kDa hGH-Y, all three of these variants are more beneficial than 22 kDa hGH-N. Although 20 kDa hGH-N (Example 1, FIG. 7) and 22 kDa hGH-V (Igout 1995) bound to the PRLR with substantially less affinity than did 22 kDa hGH-N, 20kDa hGH-V bound to PRLR very weakly, if at all, as shown in FIG. 7.

In comparison with 22 kDa hGH-N, 22 kDa hGH-V and 20 kDa hGH-N each produced less of an increase in plasma levels of the liver enzyme, alkaline phosphatase (ALP). Unexpectedly, the Inventors found that and 20 kDa hGH-V actually decreased plasma amylase levels. An increase in plasma ALP is usually an indication of liver toxicity of the drug or a liver disease in the patient. Accordingly, 22 kDa hGH-V, 20 kDa hGH-N and 20 kDa hGH-V appear to be safer alternatives for adult patients in need of GH- replacement therapy than 22 kDa hGH-N.

Additionally, compared to 22 kDa hGH-N, 20 kDa hGH-N, 22 kDa hGH-V and 20 kDa hGH-V each exhibited less adverse effects on plasma amylase concentration, with 20 kDa hGH-V having the least adverse effect, and 22 kDa hGH-V and 20 kDa hGH-N having progressively greater adverse effect on amylase, respectively. However, although 22 kDa hGH-V and 20 kDa hGH-N produce some increase in plasma amylase, those increases are smaller than that produced by 22 kDa hGH-N.

Thus, use of the 20 kDa hGH-V, 20 kDa hGH-N, and 22 kDa hGH-V can be desirable in situations in which undesirable side effects of conventional GH therapy would be harmful. Manufacture of medicaments comprising 20 kDA hGH-V, 20 kDa hGH-N, and/or 22 kDa hGH-V can lead to improved treatment of numerous conditions, and therefore can be used to decrease morbidity and mortality associated with conventional GH therapies.

Synthesis and Preparation of 20kDa hGH-V

Polypeptides of the present invention can be provided in an isolated form and in some embodiments can be purified. The term ‘isolated’ means that the material is removed from its original environment.

Polypeptides of the present invention can be derived from a naturally purified protein, a product of chemical synthesis or produced by recombinant techniques.

In one series of embodiments a polypeptide can be produced by recombinant techniques. Host cells are transformed with expression vectors and cultured in conventional nutrient media modified as appropriate for inducing promoters, selecting transformants and/or amplifying the gene(s) that produce 20 kDa hGH-V, 20 kDa hGH-N or 22 kDa hGH-V. Culture conditions such as temperature, pH and the like, are those used for the host cell selected for expression and will be apparent to those skilled in the art. It can be appreciated that for purposes of this discussion, the term “DNA,” “gene” and ‘cDNA” may be equivalent to the term “RNA” or “mRNA.” to the degree that the sequences of nucleotides in the oligonucleotides convey the information necessary to produce a polypeptide. Thus, if referring to RNA, the base uracil (U) is used, whereas if referring to DNA, the base thymine (T) is used. It can be appreciated that regardless of whether the oligonucleotide is RNA or DNA, the resulting polypeptide can be made using either type of oligonucleotide.

Examples of cloning and expression vectors for use with prokaryotic and eukaryotic hosts can be found in, for example, Sambrook et al, Molecular Cloning: A Laboratory Manual, Third Edition, Cold Springs Harbor, N. Y. (2001).

A polynucleotide (e.g., SEQ ID NO:3 or a substantial equivalent thereof) can be employed for producing a polypeptide by recombinant techniques. A polynucleotide can be included in any one of a variety of suitable vectors or plasmids for expressing a polypeptide. Such vectors include but are not limited to, chromosomal, non-chromosomal and synthetic DNA sequences e.g. derivatives of SV40, bacterial plasmids, phage DNAs, yeast plasmids, vectors derived from combinations of plasmids and phage DNAs, viral DNA such as vaccinia, adenovirus, fowl pox virus, pseudorabies and the like.

In certain embodiments, an oligonucleotide encoding 20 kD hGH-V can be expressed to produce a mRNA that can be translated into a polypeptide containing the 26 amino acids of the signal sequence of 20 kDa hHG-V (e.g., Met⁻²⁶-Ala⁻¹). Subsequent cleavage by an endopeptidase selective for internal Ala-Phe bonds can then be used to liberate the “mature” polypeptide for therapeutic use. One example of such an endopeptidase is neutral endopeptidase (E.C. 3.4.24.11), an enzyme that preferentially cleaves peptides between small aliphatic amino acids (e.g., Gly, Ala) and aromatic (Phe) or hydrophobic (e.g., Leu, Ile) amino acids. Other endopeptidases are known in the art and need not be described herein further.

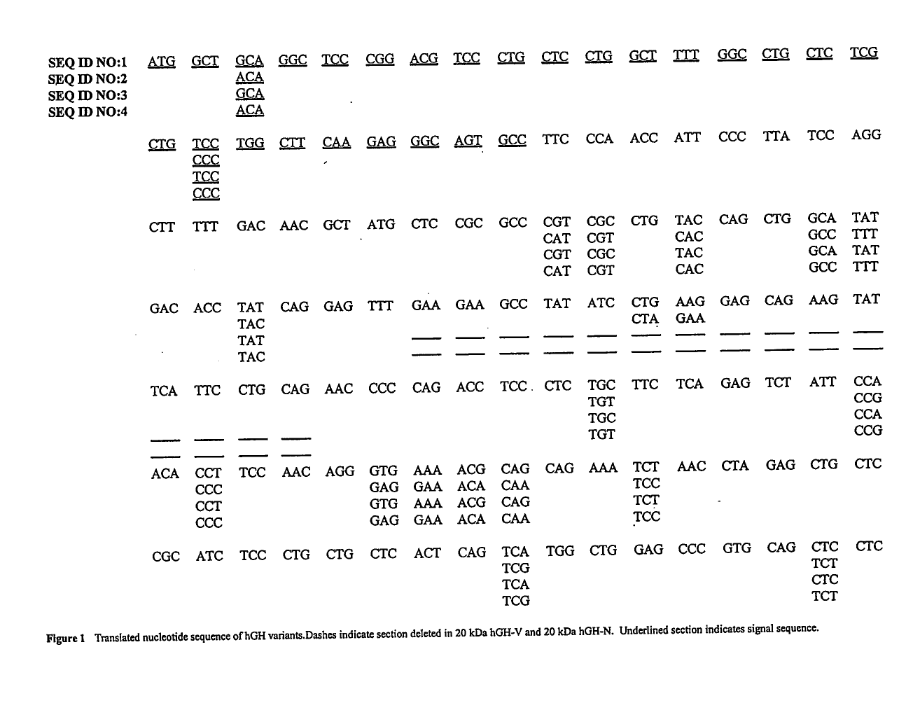

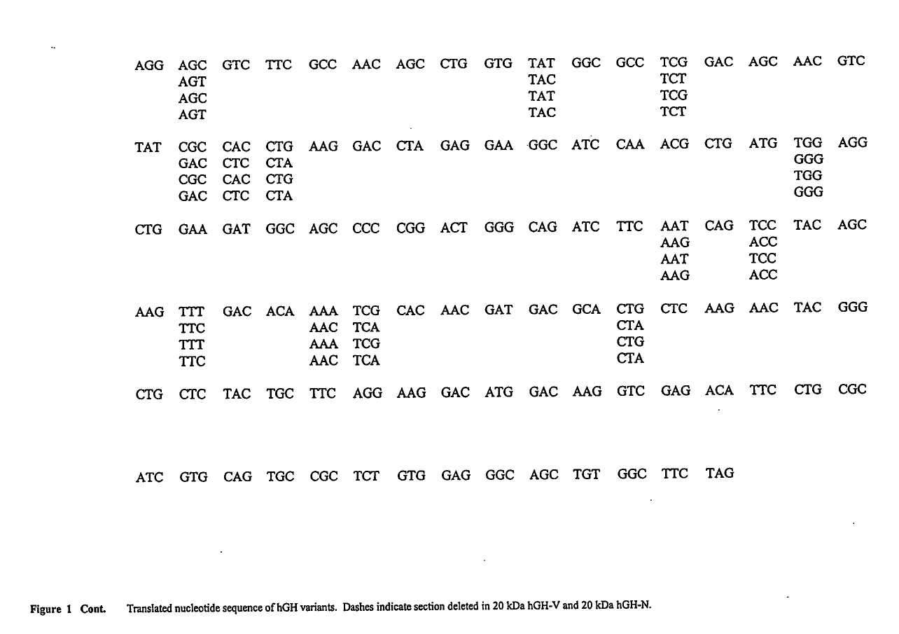

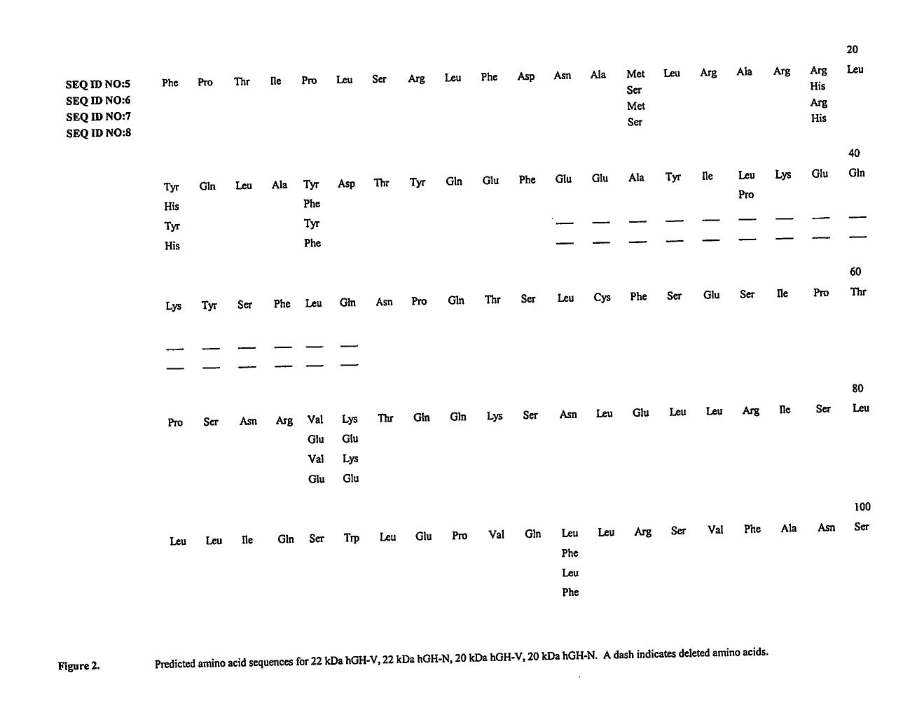

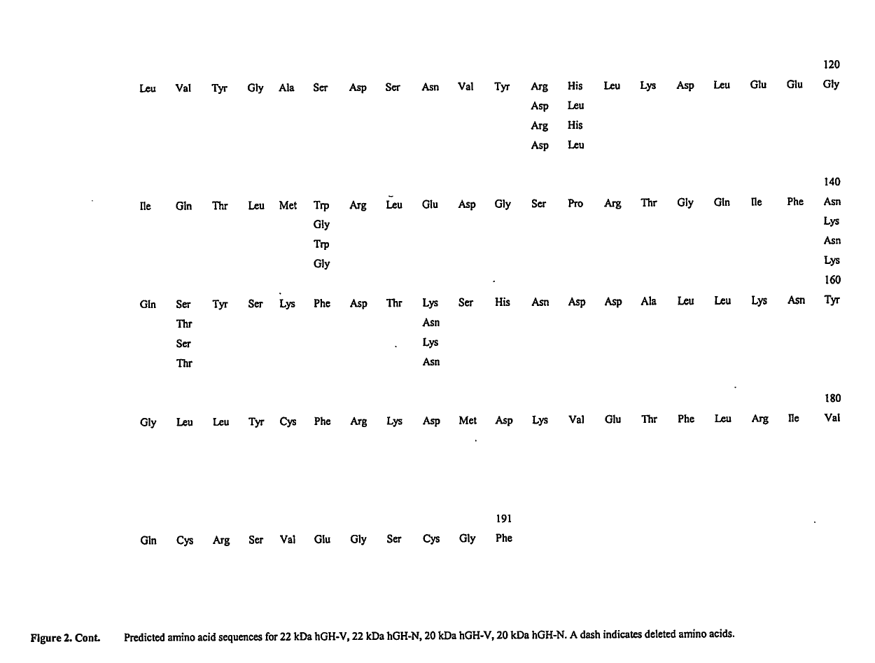

In other embodiments, mature 20 kDa hHG-V, 20 kDa hGH-N and/or 22 kDa hGH-V can be produced using an expression cassette comprising an initiation codon (ATG) followed by a codon for Phe (e.g., TTT or TTC). The remainder of the open reading frame is otherwise identical as that depicted in FIG. 1. Upon translation, the peptide can be cleaved using an aminopeptidase to remove the N-terminal Met residue, thereby producing the ‘mature’ 20 kDa hGH-V.

In still further embodiments, an expression cassette can be constructed in which a 3′ segment is added before the TTT or TTC codon for Phe¹, in which the segment encodes for a leader sequence that is normally cleaved by the cell expressing the polypeptide. Thus, the leader sequence is cleaved, producing the ‘mature’ polypeptide 20 kDa hGH-V, 20 kDa hGH-N and/or 22 kDa hGH-V for subsequent use.

Useful expression vectors for bacterial use can be constructed by inserting a structural in frame DNA sequence encoding a desired protein together with suitable translation initiation and termination signals, for example start (ATG) and stop codons, operably linked to a functional promoter. If desired, enhancer elements can also be included to increase or otherwise regulate the expression of the oligonucleotide. A vector can comprise one or more phenotypic selectable markers and an origin of replication to ensure maintenance of the vector and to, if desirable, provide amplification within the host.

Suitable vectors will be known to those skilled in the art and many are available commercially. Suitable vectors include but are not limited to bacterial vectors: pBs, pQE-9 (Qiagen), phagescript, PsiX174, pBluescript SK, pBsKS, pNH8a, pNH16a, PNH18a, pNH46a (Stratagene), pTrc99A, pKK223-3, pKK233-3, pDR540, pRIT5 (Pharmacia); eukaryotic vectors: pWLneo, pSV2cat, pOG44, pXT1, pSG (Stratagene), pSVK3, pBPV, pMSG, pSVL (Pharmacia) and the like.

An appropriate DNA sequence can be inserted into the vector by a variety of procedures. In general, a DNA sequence can be inserted into an appropriate restriction endonuclease site(s) by procedures that will be known to those skilled in the art. In one series of embodiments, restriction enzymes NcoI and HindIII can be used.

A DNA sequence in the expression vector can be operatively linked to an appropriate expression control sequence(s) (promoter) to direct mRNA synthesis. Examples of such promoters include LTR or SV40 promoter, the E. Coli lac, trp or RecA, the phage lambda PL promoter and other promoters known to control expression of genes in prokaryotic or eukaryotic cells or their viruses.

The selection of the appropriate promoter will be within the scope of those skilled in the art. Examples of promoters include but are not limited to bacterial promoters such as lacI, lacZ, T3, T7, gpt, lambda PR, trc and the like and eukaryotic promoters such as CMV immediate early, HSV thymidine kinase, early and late SV40, LTRs from retrovimses, mouse metallothionein-I and the like.

However, the above are only recited as examples, and other promoters can be used. Methods for monitoring and quantifyg expression of genes are known in the art and can be used to verify the levels of expression for producing 20 kDa hGH-V.

An expression vector can also contain a ribosome-binding site for translation initiation and a transcription terminator. A vector may also include appropriate sequences for amplifying expression (enhancers).

Mammalian expression vectors can comprise an origin of replication, a suitable promoter and enhancer and any necessary ribosome binding site, polyadenylation site, splice donor and/or acceptor sites, transcriptional termination sequences and 5′ flanking non-transcribed sequences.

In addition, an expression vector can contain a gene to provide a phenotypic trait for selection (selection marker) of transformed host cells. Suitable selection markers include dihydrofolate reductase (dfr) or neomycin resistance (neo) for eukaryotic cell culture or such as tetracycline or ampicillin resistance in E. Coli.

A vector can also include a leader sequence capable of directing secretion of translated protein into the periplasmic space, the cellular membrane or the extracellular medium.

A vector containing an appropriate DNA sequence as well as an appropriate promoter or control sequence, can be employed to transform an appropriate host to enable the host to express the protein. Suitable hosts include but are not limited to bacterial cells such as E. Coli, Bacillus subtilis, Salmonella typhimurium, various species within the genera Pseudomonas, Streptomyces, Staphlococcus Salmonella typhimurium; fungal cells such as yeast; animal cells such as COS-7 lines of monkey kidney fibroblasts and other cell lines capable of expressing a compatible vector such as the C127, 3T3, CHO, HeLa, BHK cell lines; plant cells and the like. The selection of a suitable host will be within the scope of those skilled in the art. In one embodiment the host cell is E. Coli.

Introduction of a construct into a host cell can be effected by calcium phosphate transfection, DEAE, dextran mediated transfection or electroporation (Davis et al, basic methods in Molecular Biology, 1986). In one embodiment the construct is introduced using calcium.

A host cell may be induced to express a desired protein by various methods including but not limited to tryptophan starvation, isopropylthiogalactoside (IPTG), nalidixic acid and the like. In one series of embodiments, expression can be induced by nalidixic acid.

Transcription by eukaryotic cells of a DNA encoding a polypeptide of the invention can be increased by inserting an enhancer sequence into the vector. Suitable enhancers will be known to those skilled in the art and include, but are not limited to, the SV40 enhancer on the late stage of the replication origin, a cytomegalovirus early promoter enhancer, the polyoma enhancer on the late side of the replication origin, adenovirus enhancers and the like.

It can be appreciated that workers of ordinary skill can use additional methods known in the art to produce expression systems and to use those systems to produce recombinant 20 kDa hGH-V for therapeutic purposes.

It can also be appreciated that certain host cells can be implanted directly into the subject to be treated. For example, autologous cells can be harvested from a patient or heterologous cells can be transfected with an expression vector of this invention. Such cells can then be implanted into the patient and induction of production of 20 kDa hGH-V can result in the production, in vivo, of therapeutic quantities of the 20 kDa hGH-V.

Additionally, it is contemplated that gene therapy methods, using for example, a virus such as adenovirus, or a liposome can comprise an expression cassette for expression of 20 kDa hGH-V that can be transferred in vivo into a host cell of the animal to be treated.

Isolation of 20 kDa hGH-V, 20 kDa hGH-N and 22 kDa hGH-V

Cells can be harvested by centrifugation, disrupted by physical or chemical means and a resulting crude extract can be purified. Microbial cells employed in expression of proteins can be disrupted by any convenient method including freeze-thaw cycling, sonication, mechanical disruption, use of cell lysing agents, detergents and the like.

A GH variant can be purified from recombinant cell cultures using a variety of methods, including but not limited to, ammonium sulfate or ethanol precipitation, acid extraction, anion or cation exchange chromatography, affinity chromatography, hydrophobic interaction chromatography, phosphocellulose chromatography, hydroxyapatite chromatography, lectin chromatography, gel filtration and the like.

A recombinant protein produced in bacterial culture can be isolated by initial extraction from cell pellets, followed by one or more of salting-out, aqueous ion exchange or size exclusion chromatography steps. Protein refolding steps can be used, as necessary, in completing configuration of the mature protein. SDS-PAGE and HPLC can be employed for final purification.

In other embodiments, a protein can be extracted from a bacterial culture by initially solubilizing the inclusion bodies followed by ion-exchange chromatography and gel filtration purification. The protein is then refolded using urea at high pH.

The sequence of the protein can be validated using any appropriate method including but not limited to N-terminal sequencing, proteolytic mapping and peptide sequencing. Functional characteristics can be evaluated using, for example, activation of GH receptors, immunological methods, stimulation of GH-sensitive cells in culture, and the like. In one series of embodiments, a protein can be validated by measuring the capacity of the protein to form a 1:2 complex with hGH binding protein. In other embodiments, a protein's function can be verified by its ability to activate cells transfected with GH receptors, for example, derived from a rabbit.

Depending on the host employed in a recombinant procedure to produce the polypeptide, the polypeptide of the invention may be glycosylated or non-glycosylated and may include an initial methionine amino acid (at position −1). It is known that certain prokaryotic host cells do not glycosylate proteins as well as do certain eukaryotic host cells. To promote higher degrees of glycosylation, one can provide greater levels of essential monosaccharides or their precursors into the growth medium. For example, for proteins that contain sialic acid, fucose, galactose or N-acetyl-galactosamine, a cell culture medium enriched in those nutrients can be desirably used to increase the level of expression of glycosylated forms of 20 kDa hGH-V, 20 kDa hGH-N and/or 22 kDa hGH-V. It can be readily appreciated that other sugars needed to glycosolate a GH variant can be used to supplement the growth medium as well. Moreover, if desired, one can increase the expression of a host cell's glycosyltransferases and/or nucleoside triphosphate glycosylation enzymes (sugar loading enzymes) to increase the addition of sugar residues to a 20 kDa hGH-V. Further descriptions of glycosylation can be found in Alberts et al., Molecular Biology of the Cell, Fourth Edition, Garland Science (2002).

There is some ambiguity as to the nature of the amino acid at position 14 of 20 kDa hGH-N. Martial et al (Martial et al, Science 205, 602, 1979) reported that the mRNA sequence coding for the amino acid at this position was AUG coding for methionine and Masuda et al (Masuda et al, Biophlysica Acta, 949, 125, 1988) reported that the cDNA sequence coding for the 14^{th} amino acid from the N-terminal was AGT coding for serine. While it is believed that the amino acid in this position in the 20 kDa hGH-V variant is a methionine, the invention is understood to include both variations.

Furthermore, amino acid sequences in which one or two amino acids are replaced, inserted or deleted should be understood to fall under the category of the variant 20 kDa hGH-V. Conservative variants, silent mutations and conservative amino acid substitutions should also be understood to fall under the category of the variant of this invention.

Conservative variants of nucleotide sequences include nucleotide substitutions that do not result in changes in the amino acid sequence, as well as nucleotide substitutions that result in conservative amino acid substitutions, or amino acid substitutions which do not substantially affect the character of the polypeptide translated from said nucleotides.

Pharmaceutical Compositions and Administration

GH therapy can be divided into two categories: physiological and pharmacological. Physiological therapy replacement therapy involves lower dosages. Starting replacement therapy dosages for GH in children range from 0.02 to 0.05 mg/kg per day and in adults from 0.00625 to 0.025 mg/kg per day. For a 70 kg man, the usual starting dose is 0.3 mg/day with a maintenance dose of 0.35 to 0.56 mg/day. GH replacement can be given throughout the lifetime of some patients. Pharmacologic therapy, for example to treat AIDS associated wasting, involves higher dosages; in children >1 mg/day and in adults; >1 to 3 mg/day. At this higher dosage more and more pronounced side effects can be observed.

The invention also includes a 20 kDa hGH-V, 20 kDa hGH-N and/or a 22 kDa hGH-V described herein, where the variant is conjugated to one or more water-soluble polymers in order to provide additional desirable properties of the variant while still maintaining agonist properties. Such properties include increased solubility, increased stability, reduced immunogenicity, increased resistance to proteolytic degradation, increased in vivo half-life and decreased renal clearance. Suitable polymers include, but are not limited to, polyethylene glycol, polypropylene glycol and polysaccharides. Methods of forming suitable conjugates will be known to those skilled in the art. Polyethylene glycol is particularly preferred and methods of conjugation are described in e.g. WO 95/32003.

In general, compounds of this invention can be administered as pharmaceutical compositions by one of the following routes: oral, topical, systemic (e.g. transdermal, intranasal or by suppository), parenteral (e.g. intramuscular, subcutaneous or intravenous injection), by implantation and by infusion through such devices as osmotic pumps, transdermal patches and the like. In certain embodiments, subcutaneous or intramuscular injection or injection using needle-free devices can be used, where a solution containing the compound is dispersed through the skin in a fine mist to enable subcutaneous delivery.

Compositions can take the form of tablets, pills, capsules, semisolids, powders, sustained release formulation, solutions, suspensions, elixirs, aerosols or any other appropriate compositions; and can include pharmaceutically acceptable excipients. In some embodiments, a composition is in powdered form to be reconstituted before administration or as a solution or suspension containing the GH variant. Suitable excipients are well known to persons of ordinary skill in the art, and they, and the methods of formulating the compositions, can be found in such standard references as Gennaro A R: Remington: The Science and Practice of Pharmacy, 20^{th} Ed., Lippincott, Williams and Wilkins, Philadephia, Pa. (2000). Preferred excipients include, but are not limited to, sodium chloride, phenol, m-cresol, benzyl alcohol, polysorbate 20, sodium citrate, mannitol, sodium dihydrogen phosphate, disodium hydrogen phosphate, glycine and glycerin. Suitable liquid carriers, especially for injectable solutions include sterile water, aqueous saline solution, aqueous dextrose solution and the like, with isotonic solutions being preferred for parenteral administration.

Compounds of this invention are also suitably administered by a sustained-release system. Suitable examples of sustained release compositions include semi-permeable polymer matrices in the form of shaped articles e.g. films or microcapsules. Sustained release matrices include polylactides (U.S. Pat. No. 3,773,919; EP 58,481), copolymers of L-glutamic acid and gamma-ethyl-L-glutamate, poly(2-hydroxyethyl methacrylate), ethylene vinyl acetate or poly-D-(−)-3-hydroxybutyric acid (EP 133,988). Sustained-release compositions also include a liposomally entrapped compound. Liposomes containing the compound are prepared by methods known per se: DE 3,218,121; EP 52,322; EP 36,676; EP 88,046; EP 143,949; EP 142,641; Japanese Pat. Apln. 83-118008; U.S. Pat. Nos. 4,485,045 and 4,544,545 and EP 102,324.

It can be appreciated that the above descriptions are for purposes of illustration only and are not intended to limit the scope of this invention. Rather, persons of ordinary skill can readily appreciate that modifications of the above methods and compositions can be readily used and prepared, and all such variations are considered within the scope of this invention. Further, all references cited herein are incorporated herein fully by reference.

### EXAMPLES

Other aspects of this invention are described with respect to specific examples demonstrating properties of the methods and compositions of this invention. The examples that follow are intended to illustrate advantages of this invention and are not intended to limit the scope of the invention.

### Example 1

### Ligand Binding

Materials and Methods

The study to investigate the binding interactions of hGH variants in comparison to hGH, bGH and oPRL was carried out using a well established ovine liver membrane system (Breier, B H et al., Endocrinology 135:919, 1994).

This system was chosen because the use of either ¹²⁵I-rbGH or ¹²⁵I-oPRL as radiolabelled ligands has been shown to be markers of pure somatogenic and lactogenic activity respectively. The use of ¹²⁵I-hGH is relevant for peptides with a mixture of somatogenic and lactogenic activity.

Materials

Ovine liver tissue was obtained from Romney-Dorset cross-breed castrated male lamb. The animals were in good health and were kept at high level of nutrition until slaughter. All animals were killed by barbiturate overdose, and livers were harvested within 5 min of death. The livers were dissected, washed in saline and frozen at −20° C. The experiments were approved by the animal ethics committee of the University of Auckland.

Microsomal Membrane Preparation

Ovine liver tissue was thawed at 4° C., cut into small pieces (˜1 g), and washed in cold (4° C.) 0.3 M sucrose. The tissue was then weighed (3 g maximum of liver for each tube of 45 Ti rotor). Cold 0.3 M sucrose (containing 30 μg/ml of Trasylol and 3 μg per ml of each of: pepstatin, antipain, leupeptin and benzamidine) was added to the tubes at the ratio of 1:3 w/v of initial liver weight. Homogenisation was preformed for 2 min at full speed and 0.5 min at lower speed (2.5 min total), using Janke and Kunkel homogeniser and a large homogeniser head (S25N-10G). The temperature of the homogenate was checked every 0.5 min and maintained below 10° C. The homogenate was centrifuged at 1,500×g for 20 min at 4° C., and the resultant supernatants were centrifuged sequentially at 15,000×g for 20 min and at 100,000×g (29,400 RPM, 45 Ti) for 90 minutes at 4° C. The 100,000×g pellet was incubated on ice for 20 min with 4 M MgCl₂ (to remove endogenous ligand) at the ratio of 1:2 wt/vol (ratio of initial liver weight). The preparation was then centrifuged at 125,000×g (33,000 RPM in 45 Ti rotor) and 25 mM Tris buffer was added to each tube at the ratio of 1:5 w/v of initial liver weight. The resulting pellet was suspended in 0.025 M TRIS buffer and centrifuged again at 100,000×g for 30 min at 4° C. Aliquots of the final pellet was resuspended in cold 25 mM Tris (containing all protease inhibitors mentioned in step above) at the ratio of 1 ml to 1 g of original tissue weight. The pellet was resuspended by homogenisation and further homogenised by 3 strokes in a glass/teflon homogenizer. The MMP was then aliqouted and frozen at −20° C.

Radioreceptor Assay (RRAs)

The hormone preparations for the RRAs were recombinant bovine GH, recombinant human GH-N 22 kDa, recombinant human 20 kDa hGH-N, recombinant human 22kDa hGH-V, recombinant human 20 kDa hGH-V and oPRL. All hormones were weighed out into aliquots with serial dilutions performed as required. Bovine GH was dissolved in 0.1 M NaHCO₃ (pH 8.3). Radiolabelling of the peptides was performed using the lactoperoxidase method, as previously described (Bereier et al.1988 J Endocrinol 116:169-177) and specific activities ranged from 40-50 μCi/μg. All assays were performed within 5 days of iodination and purification of the radioligands on a Sephadex G-100 column, and only fractions equivalent to monomeric ligand were used in the assays. There was no significant degradation of the radioligands during incubations of the assays. RRAs for microsomal membrane preparations and insoluble fractions were performed in triplicate and two sets of triplicates for Bo (Bₘₐₓ) tubes. The assay buffer consisted of 0.025 M TRIS, 0.01 M CaCl₂, 0.2% (wt/vol) BSA, 0.02% (wt/vol) Na azide, Trasylol at 30 μg/ml buffer and leupeptin, antipain, pepstatin and benzamidine all at 3 μg/ml buffer (pH to 7.4 with conc. HCl). The membrane preparations were incubated with unlabelled hormone, and approximately 25,000 cpm/100 μl buffer of [¹²⁵I]-rbGH or [¹²⁵I]-rhGH or [¹²⁵I]-oPRL in the incubation volume of 400 μl for 20 h at 4° C. Nonspecific binding was determined by the addition of an excess of the appropriate unlabelled ligand (10-100 μg/ml). Incubation was terminated by the addition of 2 ml of ice cold 0.025 M Tris-HCl, pH 7.4. Bound and free hormones were separated by centrifugation at 3,900 RPM at 4° C. The supernatant was discarded and the pellet was counted in a gamma-spectrometer.

Results

Assessment of Somatogenic Properties (Using ¹²⁵I-rbGH) of hGH variants in Comparison with rbGH and rhGH Using Competitive Binding Studies

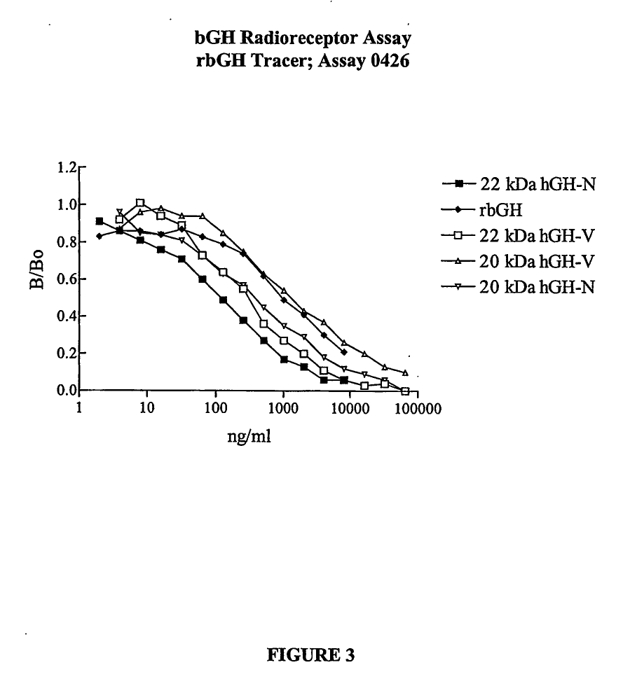

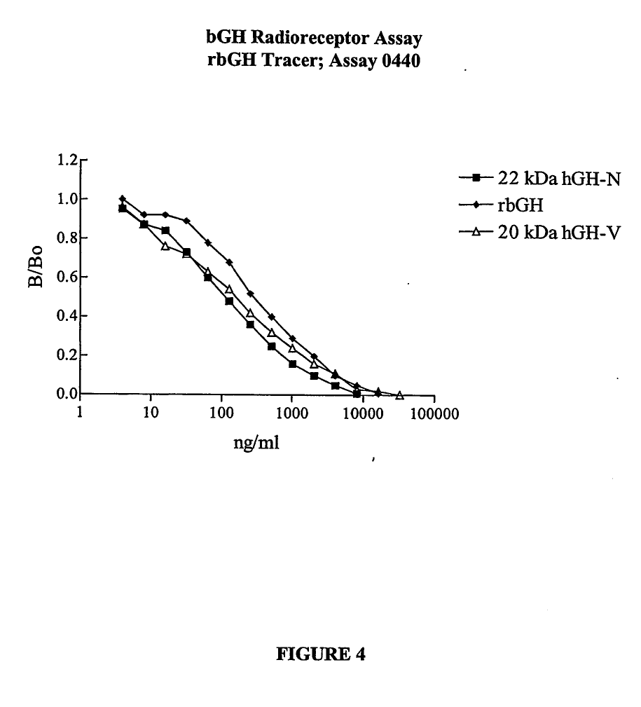

All hGH variants showed strong somatogenic potency. The displacement pattern of the binding curves was similar between all compounds. The order of somatogenic potency of the hGH variants was between that of rhGH and rbGH. The hGH variants showed quite similar somatogenic binding (FIGS. 3 and 4).

Assessment of Mixed Somatogenic and Lactogenic Properties (Using ¹²⁵I-rhGH) of hGH Variants in Comparison With rbGH and rhGH Using Competitive Binding Studies

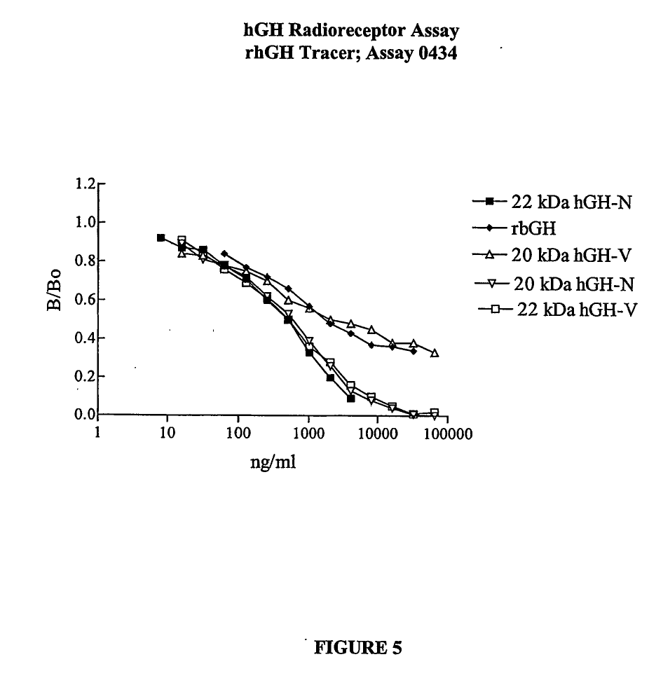

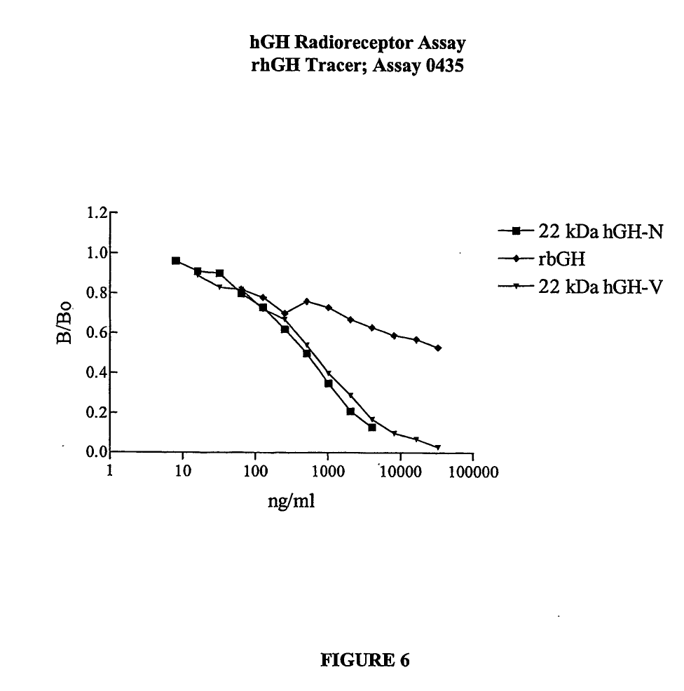

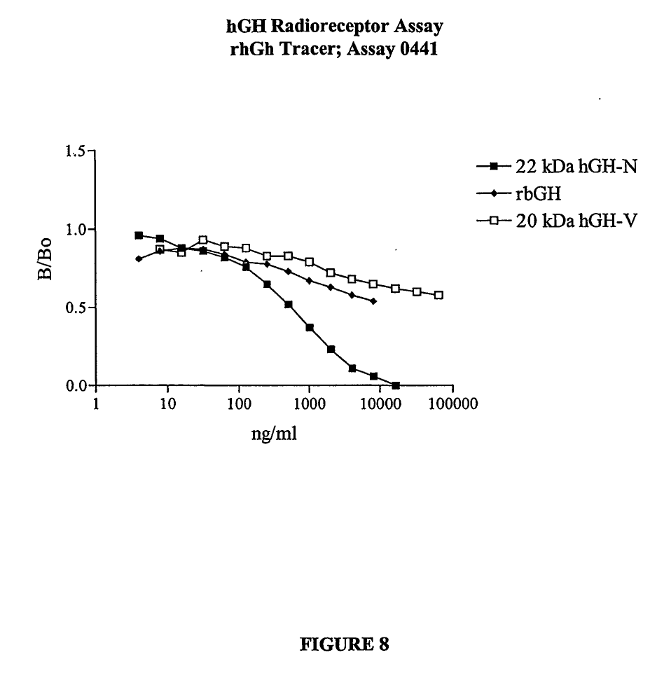

There were two distinctly different displacement curves. Firstly, the steep curve of hGH displacement indicated strong lactogenic activity. Secondly, the shallow displacement of bGH which indicated weak lactogenic activity. Most of the hGH variants followed the displacement pattern of hGH (strong lactogenic activity). Surprisingly, 20 kDa hGH-V followed the shallow bGH-ike displacement pattern indicative of weak lactogenic activity. (FIGS. 5, 6 and 8)

Assessment of Lactogenic Properties (Using ¹²⁵I-oPRL) of hGH Variants in Comparison with rbGH, Pituitary oPRL and rhGH Using Competitive Binding Studies

Assay number 0439 (FIG. 7) showed that 20 kDa hGH-V exhibited little, if any, binding interactions with ¹²⁵I-oPRL.

Interpretation of Results

20 kDa hGH-V showed a strong binding affinity with the bGH receptor, consistent with a potent somatogenic effect of that variant. However, 20 kDa hGH-V bound only weakly, if at all, to the prolactin receptor, consistent with weak lactogenic activity of that variant (FIG. 7). Based on the binding studies, we expected that biological studies would demonstrate that the 20 kDa hGH-V would exhibit growth-promoting effects and only weak lactogenic effects. These predictions were born out by the experiments described in the Example that follows.

### Example 2

### Pharmacological Studies of hGH Variants

To assess the effectiveness of the therapy comprising 20 kDa placental variant of GH, the Inventors studied effects of several GH compounds on growth, endocrine markers and and metabolic markers in an animal model of isolated growth hormone deficiency.

Experimental Procedure—Methods and Analytical Procedures

The GH-deficient dwarf (dw/dw) rat is a well-characterized model of congenital GH-deficiency. In these rats, pituitary GH is selectively reduced to about 5% of normal levels whilst other pituitary trophic hormones maintain normal secretory profiles. Other models using acquired GH-deficiency (by hypophysectomy) are confounded by the depletion of multiple pituitary hormones besides GH and also exposure of the animal to unnecessary surgical stress. Thus, use of GH-deficient dwarf rats are predictive of effects of growth hormone therapy in human conditions.

The Inventors have collected extensive baseline research data on the GH-deficient (dw/dw) dwarf rat chosen for these studies (Vickers et al., 1999; Breier et al., 1996; Gravance et al., 1997; Butler et al., 1994). The Inventors used the same colony used for our previous studies.

Male GH-deficient dwarf (dw/dw) rats were purchased from a colony maintained by the Animal Resources Unit at the University of Auckland (ethics approval No. R38) at a weaning age (21-22) days. Animals were acquired at this age (i.e. 3-4 weeks prior to investigative age) to allow time for acclimatisation and familiarisation of handling with the investigating personnel. Animals were housed in a dedicated facility using standard rat cages, normal light-dark cycles and unlimited access to food and water. The animals were monitored daily from weaning until the completion of the studies.

At 7-8 weeks of age, male GH-deficient dwarf rats were weight-matched and assigned to one of 6 treatment groups (n=6) to receive either vehicle (physiological saline (0.9%))) or GH. The treatment groups were as follows:

| Group | Dose |
|---|---|
| Saline | 0 |
| hGH (Genotropin) | 1.0 ug/g/day |
| bGH (Monsanto) | 1.0 ug/g/day |
| Pituitary 20 kDa hGH | 1.0 ug/g/day |
| Placental 20 kDa hGH | 1.0 ug/g/day |
| Placental 22 kDa hGH | 1.0 ug/g/day |

Test Compounds

hGH was reconstituted using physiological saline (0.9%). bGH was reconstituted using carbonate buffered saline (pH 9.4). The hGH variants were reconstituted in sterile water at pH 11.0. Compounds were dissolved fresh on the day of injection. Injections (volume 100 ul) were administered by subcutaneous injection given twice daily at 0800 and 1700 h using a fine gauge diabetic syringe (29 g). Animals were treated for 7 days with the last injection administered on the morning of day 8 following an overnight fast. Animals were sacrificed on the morning of day 8 following the final GH injection.

Observations

Body Weights

Animals were weighed between 8-9 am every day for the duration of the experiment. Individual animals were observed daily for any signs of clinical change, reaction to treatment or ill health. There were no indications whatsoever of any adverse stress responses and related symptoms in any of the treatment groups.

Food Consumption

Food intake was measured on a daily basis for the duration of the trial. Relative food intake per rat (grams consumed per gram body weight per day) was calculated using the amount of food given to and the amount of food left uneaten by each pair in each treatment group.

Water Consumption

Water consumption was calculated daily by weighing water bottles at the same time on each day of the study.

Body Length

Body lengths (nose-anus and nose-tail) and bone length (tibial) were assessed post-mortem using standard measurement techniques and also by use of peripheral quantitative computed tomography (PQCT, Stratec) analysis. Bone density was also assessed via PQCT.

Tissue Measurements

On day 8, following an overnight fast, animals were sacrificed by halothane anaesthesia followed by decapitation. Measurements of body length, carcass weight, organ weights (liver, spleen, kidney, adrenals, heart, and pituitary) and fat pad weight (retroperitoneal) were recorded.

Plasma Measurements

Blood samples were collected following an overnight fast. Trunk blood was collected from animals following decapitation under halothane anaesthesia. Samples were collected into heparinised tubes and centrifuged for harvesting of plasma. Blood samples were analysed for insulin*, glucose, FFAs, leptin*, IGF-I, glycerol, triglycerides, cholesterol, markers of hepatic function (ALT, AST, ALP), and for markers of protein synthesis.

Plasma FFAs, triglycerides and glycerol were measured by diagnostic kit (Boehringer-Manmheim #1383175 and Sigma #337 respectively). Plasma IGF-I was measured by RIA as described previously. Plasma glucose concentrations were measured using a calorimetric plate assay. All other plasma analytes (Giver enzymes, electrolytes) were measured by a BM/Hitachi 737 analyser by Gribbles Veterinary pathology (Auckland, New Zealand).

Data Analysis

Data was analysed by one-way factorial ANOVA with post-hoc correction (factor=treatment). Growth rates were also analysed via repeated measures. Body fat was also analysed by ANCOVA with body weight as a covariate.

Previous data provided the basis of power calculations for the proposed studies (assuming α=0.05). For insulin sensitivity, an n of 10 will detect with a power of 80% a change of 0.2 and at 95% a change of 0.26 ng/ml with an SD of 0.15 ng/ml. For body length, an n of 10 will detect with a power of 80% a change of 6.88 mm and at 95% a change of 7.97 mm with an SD of 5.2 mm

Results

Body Weights

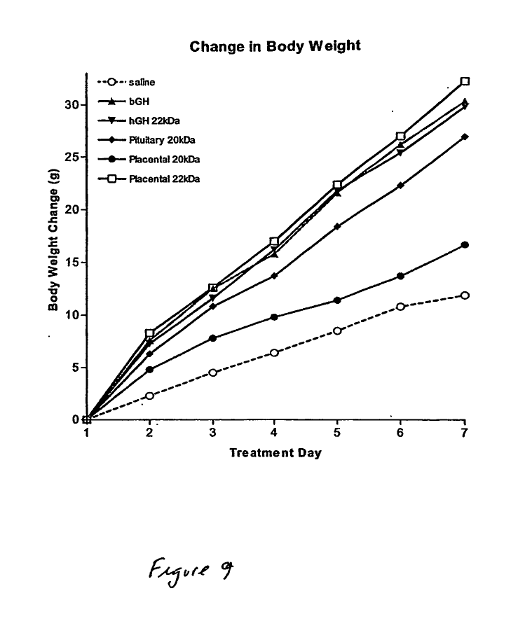

Body weights were significantly increased in all treatment groups compared to saline (FIGS. 9, 10A and 10B). Degrees of statistical significance are provided below.

| | |
|---|---|
| bGH versus saline | p < 0.0001 |
| hGH versus saline | p < 0.0001 |
| Placental 22 kDa versus saline | p < 0.0001 |
| Pituitary 20 kDa versus saline | p < 0.0001 |
| Placental 20 kDa versus saline | p < 0.005 |

There were no statistically significant differences in total body weight gain between animals treated with bGH, hGH or the 22 kDa placental GH variant. Animals treated with the 20 kDa placental variant showed significantly reduced weight gain compared to all other GH treated groups. Animals treated with the 20 kDa pituitary variant exhibited less weight gain than animals treated with bGH and the placental 22 kDa variant (hGH versus pituitary 20 kDa; p=0.07).

FIG. 10B shows that with all GH variants tested, there was an initial increase in weight gain, followed by a partial return toward the changes observed with saline. However, for the 20 kDa placental GH variant, the initial weight gain over the first 2 days of treatment was followed by a return to daily weight gain similar to that of saline treated animals. The bGH and pituitary 20 kDa treated animals appeared to show a slight rebound in weight gain at day 4. hGH and placental 22 kDa treated animals showed a marked initial weight gain followed by a constant daily weight gain for the remainder of the trial.

Tibial Length

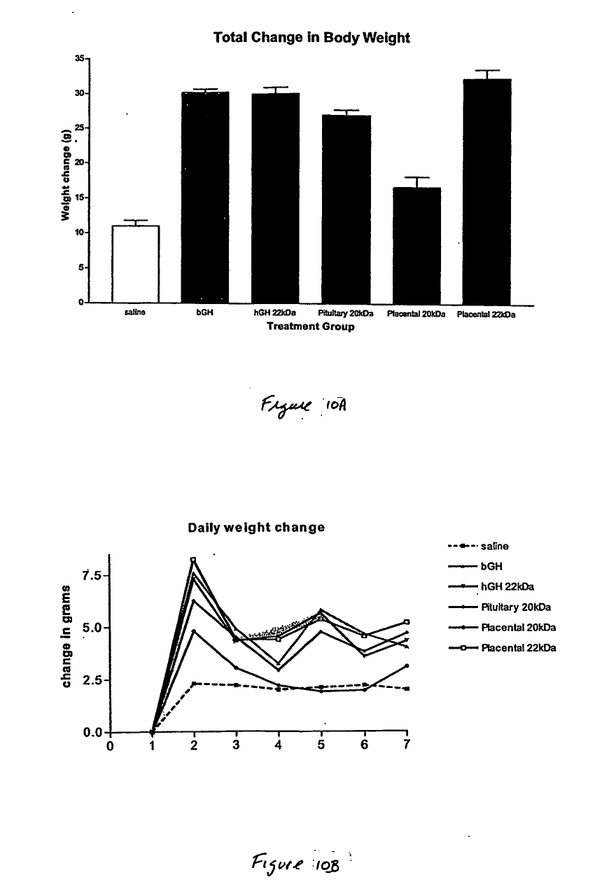

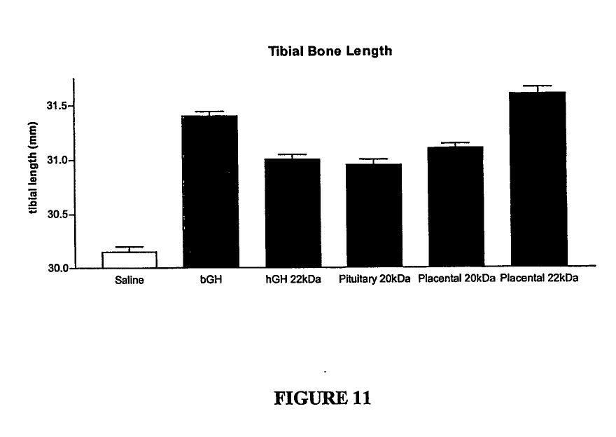

Tibial bone length was slightly but significantly increased in all treatment groups compared to saline controls (FIG. 11). No statistically significant difference was observed between any of the GH treatment groups in tibial bone length. Total bone area and cortical bone area were significantly increased in animals treated with the 20 kDa pituitary GH variant. No changes in bone density were observed in any of the treatment groups. Indices of bone strength, as measured by the stress strain index (SSI), were increased in animals treated with bGH, pituitary 20 kDa GH and the placental variants compared to saline controls.

Body Length

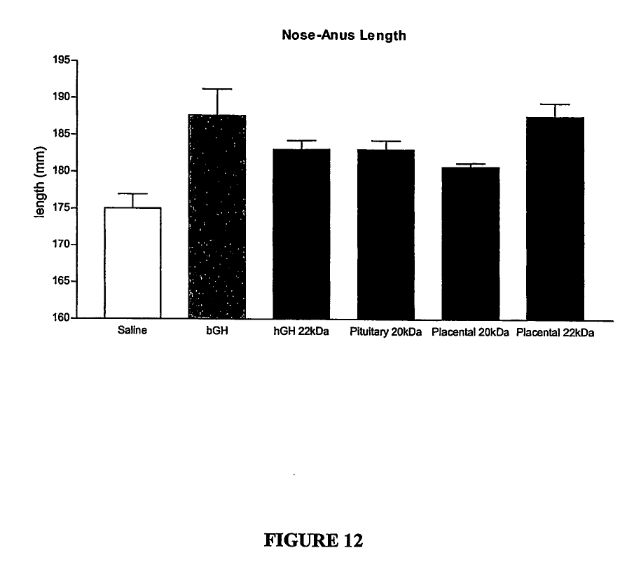

Nose anus lengths were increased in all treatment groups compared to saline controls. The increases were statistically significant in all groups with the exception of the 20 kDa placental variant which exhibited a strong trend towards increased body length that approached statistical significance (p=0.0576) FIG. 12).

Food Intake

Total food intake was increased compared to controls in animals treated with bGH, hGH and placental 22 kDa variant (FIG. 13A; top). Food intake was also significantly different between the 20 kDa and 22 kDa placental variant treatment groups. However, when food intake was adjusted for changes in body weight, no significant differences in relative food intake were observed although a trend towards increased appetite was apparent in the 22 kDa placental variant treatment group (p=0.1) (FIG. 13B; bottom).

Water Intake

Water intake was not significantly different between any of the treatment groups compared to saline controls for the duration of the trial.

Tissue Weights

Tissue weights are analysed as a percentage body weight unless otherwise stated.

Retroperitoneal Fat Depot

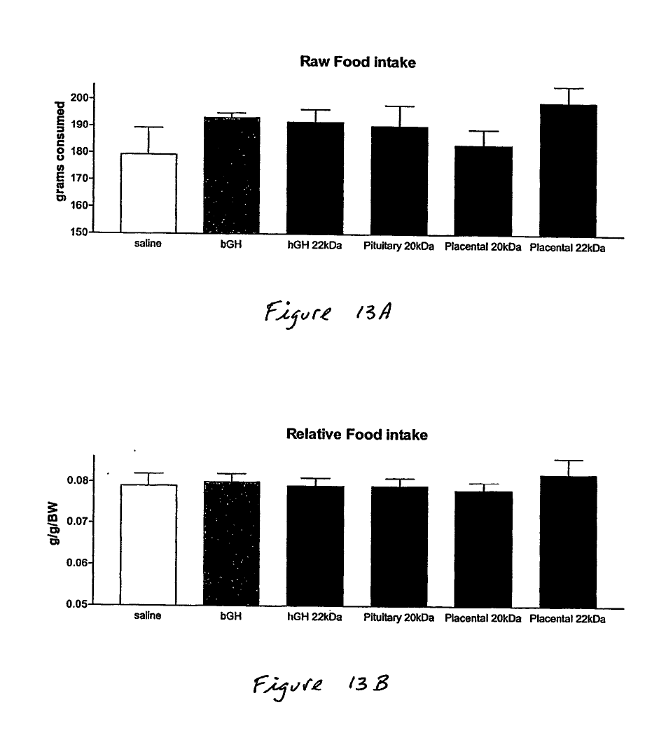

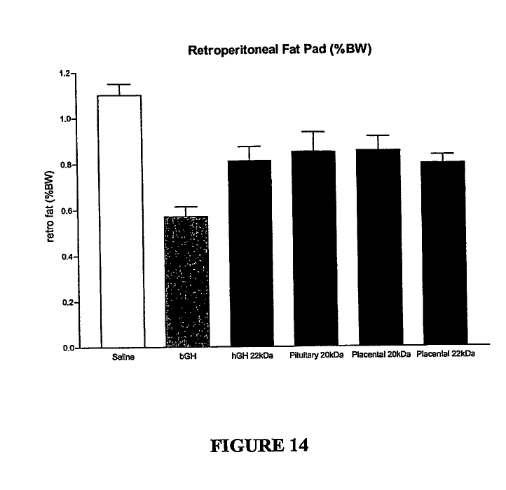

Retroperitoneal fat pad weight was significantly reduced in all GH treatment groups compared to saline controls (FIG. 14). bGH was significantly more lipolytic than each of the hGH variants. The response between the placental variants was not significantly different (placental 20 kDa versus placental 22 kDa p =0.53.

Liver

Relative liver size was increased in animals treated with the 22 kDa placental variant compared to saline treated animals. Liver size was relatively decreased in animals treated with the 20 kDa placental variant compared to saline treated animals.,

Spleen

Relative spleen weight was increased in all GH treatment groups compared to saline controls, most marked in the placental 22 kDa placental GH variant treated animals.

Heart

There were no significant differences in heart size between any of the GH treatment groups and saline controls. There was a trend towards an increased heart size in animals treated with the 20 kDa placental GH variant (p=0.09 versus saline).

Kidney

Kidney size was not affected by any treatment compared to saline controls.

Adrenal Glands

Adrenal gland size was significantly increased compared to controls in animals treated with bGH, pituitary 20 kDa GH, placental 20 kDa and 22 kDa variants. Adrenal size was not affected by treatment with hGH (p=0.1551).

Brain

Relative brain weight was decreased in animals treated with the 22 kDa placental GH variant compared to saline treated animals and those treated with the 20 kDa placental variant or hGH.

Testes

Relative testes size was increased compared to controls in animals treated with bGH, hGH, pituitary 20 kDa hGH and the 22 kDa placental variant. Treatment with the placental 20 kDa variant had no effect on relative teste size (p=0.83).

Plasma Measurements

IGF-I

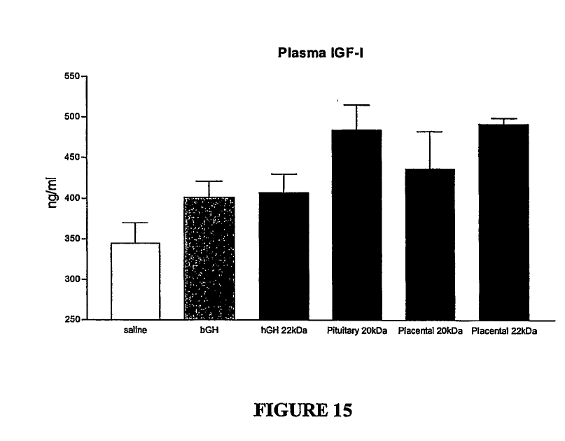

Plasma IGF-I was increased by all GH compounds (FIG. 15). The increases were statistically significant in the pituitary 20 kDa, placental GH 20 kDa and placental 22 kDa GH variant treatment groups. Treatment with bGH or hGH at 1.0 mg/kg/day did not significantly elevate plasma IGF-I concentrations (p=0.17 and p=0.13 respectively). Placental 22 kDa GH elevated plasma IGF-I levels significantly above that of hGH or bGH treatment

Glucose

There was no effect of any of the treatment groups on fasting plasma glucose concentrations.

Free Fatty Acids (FFAs)

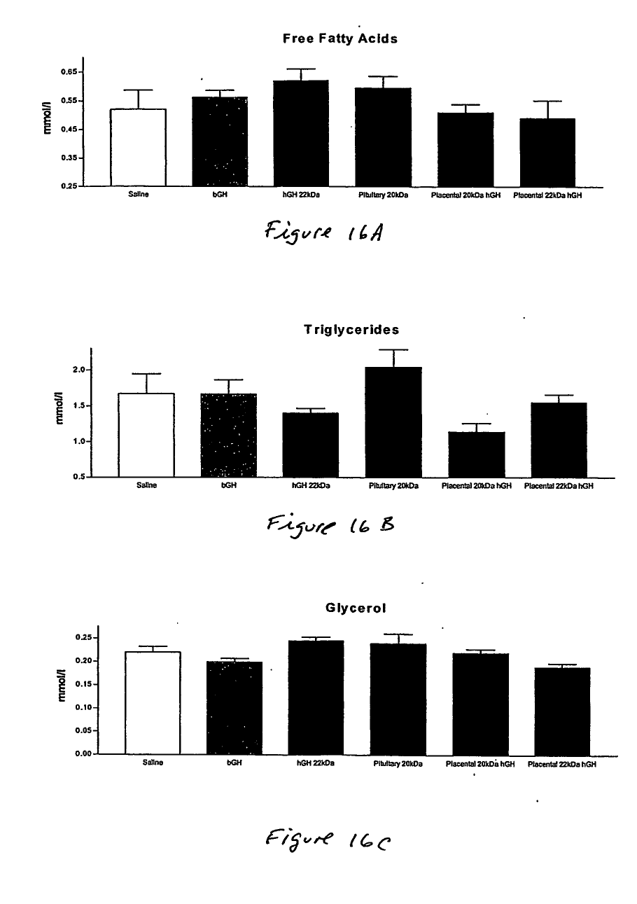

There was no statistically significant effect of any of the treatment groups on fasting plasma free fatty acid concentrations, although there was a trend toward slight increases in FFAs with 22 kDa hGH and Pituitary 20 kDa HG, and a slight reduction in response to placental 20 kDa hGH or placental 20 kDa GH (FIG. 16A; upper graph).

Triglycerides

Placental 20 kDa GH significantly decreased fasting plasma triglyceride concentrations compared to saline controls or animals treated with either pituitary 20 kDa or bGH (FIG. 16B; middle graph).

Glycerol

There was no effect of any of the treatment groups on plasma glycerol concentrations compared to saline controls (FIG. 16C; bottom graph). Plasma glycerol was significantly elevated in hGH and pituitary 20 kDa animals compared to the bGH treatment group. Concentrations were significantly lower in the placental 22 kDa treatment group compared to hGH treated and pituitary 20 kDa GH treated animals.

Biochemical Markers

Alkaline Phosphatase (ALP)

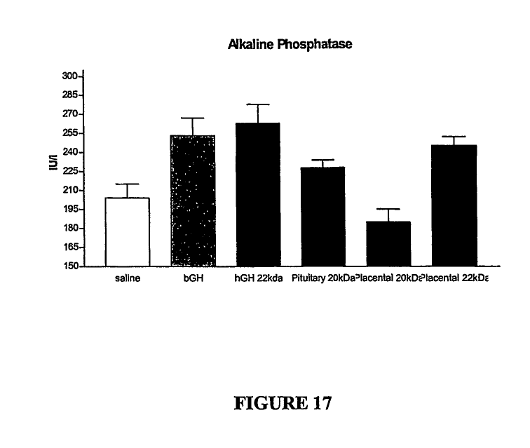

Plasma ALP was significantly increased in animals treated with bGH, hGH or the 22 kDa placental GH variant (FIG. 17). There was no effect of pituitary 20 kDa or the placental 20 kDa variant on ALP concentrations (p=0.16 and 0.26 respectively).

Sodium

Plasma sodium concentration was significantly increased in animals treated with hGH compared to saline controls and a strong trend was seen in bGH treated animals towards elevated sodium concentrations (p=0.06 versus saline). The placental GH variants and the pituitary 20 kDa GH had no effect on plasma sodium.

Creatine Kinase (CK)

Plasma CK was significantly decreased in animals treated with placental 20 kDa GH compared to saline controls and was significantly lower in this group compared to animals treated with bGH or pituitary 20 kDa GH (saline versus hGH p=0.08, versus placental 22 kDa p=0.2).

Potassium

Plasma potassium concentratnion was significantly increased in the pituitary 20 kDa and hGH treatment groups compared to saline controls. bGH and the placental GH variants had no effect on plasma potassium concentrations.

Bilirubin

Plasma bilirubin concentration was not significantly altered by any treatment group compared to saline controls.

Aspartate Aminotransferase (AST)

AST concentrations were significantly decreased in animals treated with bGH compared to saline controls. None of the hGH variants had any significant effect on plasma AST.

Globulin

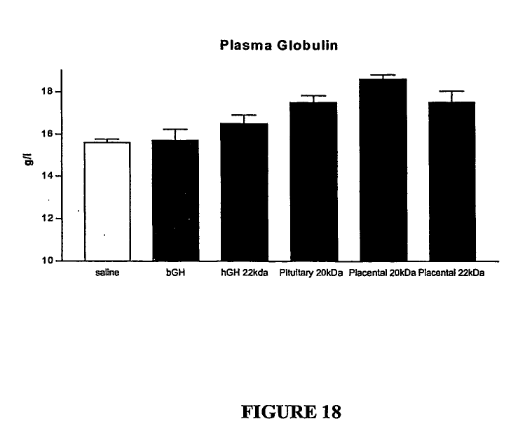

Plasma globulin was significantly increased in animals treated with the placental variants and with the 20 kDa pituitary GH (FIG. 18). Treatment with bGH or hGH had no significant effect on plasma globulin. Increased gamma globulin may indicate multiple myeloma, chronic inflammatory disease, hyperimmunization, acute infectdion or Waldenstrom's macroblobulinemia.

Creatinine

There were no differences in plasma creatinine between any of the treatment groups.

Urea

There was no significant effect of any of the GH treatments versus saline controls on plasma urea concentrations.

Amylase

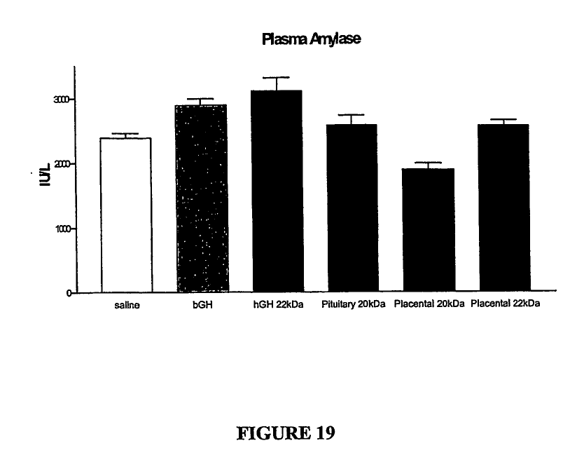

Plasma amylase concentrations were significantly increased in animals treated with bGH or hGH compared to saline controls (FIG. 19). Treatment with the placental 20 kda variant significantly lowered plasma amylase concentrations compared to saline treated animals.

Because amylase is produced by glands (e.g., pancreatic exocrine cells, parotid salivary glands), its presence in the plasma at abnormally high levels may indicate damage to the pancreas or salivary glands, permitting the enzyme to leak into the interstitial fluid and thereafter into the plasma. Thus, increased plasma amylase may indicate acute pancreatitis, pancreatic cancer, cholecystitis, ectopic or ruptured tubal pregnancy, mumps, intestinal obstruction, macroamylesia, obstruction of the pancreatic duct or bile duct, or a perforated ulcer.

Alanine aminotransferase (ALT)

Plasma ALT concentrations were significantly decreased in animals treated with bGH or the placental 20 kDa GH compared to saline controls.

Lipase

There were no significant differences in plasma lipase concentrations between any of the treatment groups.

Total Protein

There were no significant effects on plasma total protein concentrations compared to saline controls. Total protein concentrations were significantly lower in the 22 kDa placental GH group compared to animals treated with hGH.

Total Cholesterol

Total plasma cholesterol concentrations were increased in the 20 kDa placental GH group compared to saline controls. There was a minor yet significant difference in cholesterol concentrations between the 22 kDa placental and 20 kDa placental GH variants (22 kDa 2.9±0.1 mmol/l, 22 kDa 2.5±0.1 mmol/l, p=0.05).

Albumin

Plasma albumin concentrations were slightly but significantly reduced in the animals treated with the pituitary 20 kDa GH or the placental 20 kDa and 22 kDa variants. Plasma albumin concentrations were not significantly altered with bGH or hGH treatment.

Because albumin is synthesized by the liver, decreased serum albumin may result from liver disease. It can also result from kidney disease, which allows albumin to escape into the urine. Decreased albumin may also be explained by malnutrition or a low protein diet. Lower-than-normal levels of albumin may indicate ascites (fluid in the peritoneal cavity, glomeriulohephritis (kidney disorder of filtration), liver disease (hepatitis, cirrhosis, hepatocellular necrosis), malabsorptive conditions (e.g., Crohn's disease, sprue, Whipple's disease), malnutrition or nephrotic syndrome.

Sodium/Potassium Ratio (Na/K ratio)

The plasma Na/K ratio was significantly decreased in animals treated with hGH compared to saline controls and a trend was observed in the pituitary 20 kDa group (p=0.06 versus saline).

Chloride

Plasma chloride concentrations were significantly increased in animals treated with hGH and bGH compared to saline controls and a strong trend in the 20 kDa placental GH group (p=0.06 versus saline).

Hematocrit

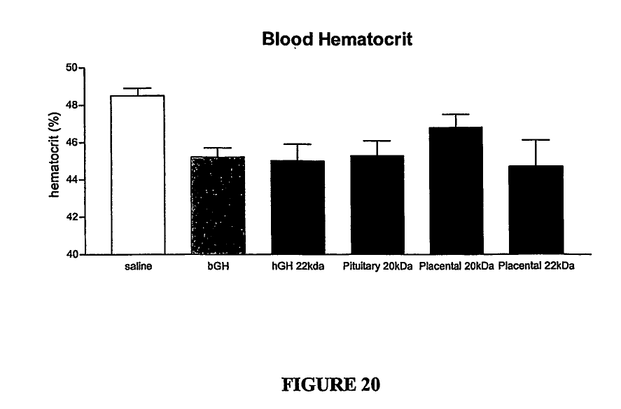

Fasting blood hematocrit was significantly decreased in all treated animals. The decrease in hematocrit was not as marked in animals treated with the 20 kDa placental variant as compared to the other treatment groups (FIG. 20). Statistical significance is shown below.

| | |
|---|---|
| Saline versus bGH | p = 0.0003 |
| hGH | p = 0.0008 |
| Pituitary 20 kDa | p = 0.0004 |
| Placental 22 kda | p = 0.042 |

Discussion

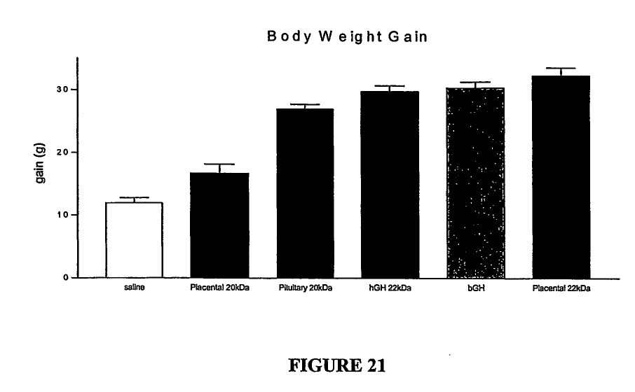

All compounds tested produced significant weight gain above that of saline treated animals. In particular, 20 kDa hGH-V exhibited significant somatogenic effects, and therefore is a suitable therapeutic agent for treatment of disorders that have conventionally been treated with GH or other variants. The weight gain in the 20 kDa hGH-V group, after an initial peak after 48 hours, returned to parallel that of saline treated animals for the remainder of the trial (FIG. 21). Of note however, is that the somatogenic 20 kDa hGH-V caused a increment in tibial bone length and nose anus length that was not statistically significant from the effects on other GH treatment groups.

Although the mechanisms of action of 20 kDa hGH-V are not known with certainty, it is possible that the reduced body weight gain in the 20 kDa hGH-V group, rather than being an indication of a weaker somatogenic properties of the 20 kDa hGH-V, may be associated with the decreased water retentive properties of the compound in comparison with other treatments. A well-characterised effect of GH treatment is increased plasma volume (Johannsson et al, 2002). Although blood hematocrit was reduced in all treatment groups, the increase in plasma volume in the 20 kDa placental GH group was less marked than in the other treatment groups. Plasma sodium was significantly increased in hGH treated animals compared to saline controls but was not affected by any other treatment.

20 kDa placental GH variant was as lipolytic as any of the other hGH variants.

Interestingly, plasma IGF-I was increased in the animals treated with bGH or hGH. Although a strong trend towards an increase was apparent, the failure to reach statistical significant may be a result of relatively low dose, duration or even buffering systems. Spleen weights were slightly but significantly increased in all GH treatment groups which is a normal observation following GH treatment in rodents. Although cardiac hypertrophy is often observed with GH treatment, in the present study no increase in heart size was observed although a trend was observed towards increased relative heart size in the 20 kDa placental GH treated animals. Relative adrenal size was also increased in all treatment groups with the exception of those treated with hGH.

Therefore, we conclude from the studies described herein the following. First, 20 kDa hGH-V, 22 kDa hGH-V and 20 kDa hGH-N are effective somatogenic agents suitable for treating conditions conventionally treated with other GH compounds. Although the exact mechanisms are not known with certainty, the finding that 20 kDa hGH-V, 20 kDa hGH-N and 22 kDa hGH-V bound to GH receptors in hepatic microsomes strongly suggests that the mechanisms for somatogenic effects are similar to those used by conventional GH compounds.

Importantly, we unexpectedly found that the somatogenic effects of 20 kDa hGH-V were not associated with the typical adverse side effects often observed after conventional GH therapy. We also found that other GH variants, namely 20 kDa hGH-N and 22 kDa hGH-V produced lesser undesirable side effects than conventional therapy with 22 kDa hGH-N. In particular, there was reduced evidence of damage to at least the liver and the pancreas.

Conventional GH compounds bound to prolactin receptors, and that mechanism may be responsible for lactogenic effects of conventional therapeutics. In contrast, 20 kDa hGH-V bound prolactin receptors only weakly, if at all, indicating that lactogenic effects are not likely to be found in biological or biochemical assays. The other variants, 20 kDa hGH-N and 22 kDa hGH-V bound to prolactin receptors with less affinity than 22 kDa hGH-N, suggesting that 20 kDa hGH-N and 22 kDa hGH-V have less side effects than 22 kDa hGH-N. These conclusions were confirmed by working examples in vivo that demonstrated that markers for liver toxicity (e.g., alkaline phosphatase) were not increased by 20 kDa hGH-V, were increased somewhat by 20 kDa hGH-N and 22 kDa hGH-V, but were increased more by the conventional GH therapeutic agent 22 kDa hGH-N. Further, we observed no evidence of pancreatic or salivary gland damage because plasma amylase levels were not increased by 20 kDa hGH-V. This is in striking contrast to the increased plasma amylase levels associated with conventional GH therapy with 22 kDa hGH-N. Finally, 20 kDa hGH-N and 22 kDa hGH-V showed side effects of intermediate magnitude, between the lack of side effects of 20 kDa hGH-V and the well known side effects of 22 kDa hGH-N.

Therefore, the inventors have discovered that variants of human growth hormone, namely 20 kDa hGH-V, 20 kDa hGH-N and 22 kDa hGH-V can be useful therapeutic agents in situations in which conventional growth hormone therapy would lead to undesirable side, effects. Further, 20 kDa hGH-V, 20 kDa hGH-N and 22 kDa hGH-V can be used in the manufacture of medicaments for treating disorders for which growth hormone therapy is desired.

### REFERENCES

All of the references cited herein are incorporated fully by reference, including those in the list below.
Ader M, Agajanian T, Finegood D T, Bergman R N (1987) Endocrinology 120, 725-731.
Asada N, Takahashi Y, Wada M, Naito N, Uchida H, Ikeda M, Honjo M (2000) Mol. Cell. Endocrinol. 162, 121-129.
Boguszewski C L, Svensson P-A, Jansson T, Clark R, Carlsson L M S, Carlsson B (1998) J. Clin. Endocrinol. Metab. 83, 2878-2885.
Bole-Feysot C, Goffin V, Edery M, Binart N and Kelly P. 1998 Endocrine Reviews19(3); 225-268.
Breier B H, Funk B, Ambler G R, Surus A, Waters M J and Gluckman P D. 1994. Endocrinology 135: 919-928.
Breier B H, Gluclknan P D, Bass J J. 1988 The somatotrophic axis in young steers: influence of nutritional status and oestradiol-17beta on hepatic high- and low-affinity somatotrophic biding sites. J Endocrinol 116:169-177.
Breier B H, Vickers M H, Gravance C G, Casey P J. (1996) Endocrinology. 137(9):4061-4.
Butler A A, Ambler G R, Breier B H, LeRoith D, Roberts C T Jr, Gluckman P D. (1994) Mol Cell Endocrinol. 101(1-2):321-30.
Cooke N E, Ray J, Watson M A, Estes P A, Kuo B A, Liebhaber S A (1988) J Clin. Invest. 82, 270-275.
Cutfield W S, Wilton P, Bennmarker H, Albertsson-Wikland K, Chatelain P, Ranke M B, Price D A. (2000) The Lancet 355, 610-13.
Daugaard J R, Laustsen J L, Hansen B S, Richter E A.(1999) J Endocrinol. 160(1):127-35.
Estes P A, Cooke N E, Liebhaber S A (1992) J. Biol. Chem. 267, 14902-14908.
Frigeri L G, Peterson S M, Lewis U J (1979) Biochem. Biophys. Res. Commun. 91, 778-782.
Goodman H M, Tai L R, Ray J, Cooke N E, Liebhaber S A (1991) Endocrinology 129, 1779-1783.
Gravance C G, Breier B H, Vickers M H, Casey P J. (1997) Anim Reprod Sci. 49(1):71-6.
Hsiung H M, Mizushima S (1988) Biotechnol. Genet. Eng. Rev. 6, 43-65.
Igout A, Frankenne F, L'Hermite-Balériaux M, Martin A, Hennen G (1995) Growth Regul. 5, 60-65.
Ishikawa M, Hiroi N, Kamioka T, Tanaka T, Tachibana T, Ishikawa H, Miyachi Y (2001) Eur. J. Endocrinol. 145, 6, 791-797.
Ishikawa M, Tachibana T, Kamioka T, Horikawa R, Katsumata N, Tanaka T (2000) Growth Horm. IGF Res. 10, 4, 199-206.
Johannsson G, Sverrisdottir Y B, Ellegard L, Lundberg P A, Herlitz H. (2002) J Clin Endocrinol Metab. 87(4):1743-9.
Juarez-Aguilar E, Castro-Muniozledo F (1995) Biochem. Biophys. Res. Comwiun. 217, 28-33.
Kostyo J L, Cameron C M, Olsen K C, Jones A J S, Pai R C (1985) Proc. Natl. Acad. Sci. 82, 4250-4253.
MacLeod J N, Worsley I, Ray J, Friesen H G, Liebhaber S A, Cooke N E (1991) Endocrinology 128, 1298-1302.
Martin B C, Warram J H, Krolewski A S, Bergman R N, Soeldner J S, Kahn C R. (1992) Lancet 340, 925-9.
Ray J, Okamura H, Kelly P A, Liebhaber S A, Cooke N E (1990) J. Biol. Chem. 265, 7939-7944.
Ricón-Limas D E, Resndez-Prez D, Ortíz-López R, Alvidrez-Quihui AE, Castro-Muñozledo F, Kuri-Harcuch W, Martónez-Rodróguez H G, Barrera-Saldaña H A (1993) Biochim. Biophys. Acta 1172, 49-54.
Satozawa N, Takezawa K, Miwa T, Takahashi S, Hayakawa M, Ooka H (2000) Growth Horm. IGF Res. 10, 187-192.
Takahashi S, Satozawa N (2002) Horm. Res. 58, 157-164.
Tsunekawa B, Wada M, Ikeda M, Uchida H, Naito N, Honjo M (1999) Endocrinology 140, 3909-3918.
Uchida H, Naito N, Asada N, Wada M, Ikeda M, Kobayashi H, Asanagi M, Mori K, Fujita Y, Konda K, Kusuhara N, Kamioka T, Nakashima K, Honjo M (1997) J. Biotechnol. 55, 101-112.
Vickers M H, Casey P J, Champion Z J, Gravance C G, Breier B H. (1999) Growth Horm IGF Res. 9(4):236-40.
Wada M, Uchida H, Ikeda m, Tsunekawa B, Naito N, Banba S, Tanaka E, Hashimoto Y, Honjo M (1998) Mol. Endocrinol. 12,146-156.
Wang D, Sato K, Demura H, Kato Y, Maruo N, Miyachi Y (1999) Endocrine J 46, 1, 125-132.

We claim:

1. A method of treating a condition in a mammal, comprising administering to said mammal a pharmaceutically effective amount of 20 kDa hGH-V or a polypeptide that is substantially identical to 20 kDa hGH-V.

2. The method of claim 1, wherein said condition is adult-onset growth hormone deficiency.

3. The method of claim 1, wherein said condition is childhood-onset growth hormone deficiency.

4. The method of claim 1, wherein said condition is cystic fibrosis.

5. The method of claim 1, wherein said condition is osteoporosis.

6. The method of claim 1, wherein said condition is skeletal dysplasia.

7. The method of claim 1, wherein said condition is chronic kidney failure.

8. The method of claim 1, wherein said condition is depression.

9. The method of claim 1, wherein said condition is memory loss.

10. The method of claim 1, wherein said condition is a catabolic state.

11. The method of claim 1, wherein said condition is anorexia.

12. The method of claim 1, wherein said condition is hypertension.

13. A pharmaceutical composition comprising a 20 kDa hGH-V and a pharmaceutically acceptable excipient.

14. A pharmaceutical composition comprising a 20 kDa hGH-V, a pharmaceutically acceptable excipient and a binder.

15. A pharmaceutical composition comprising a 20 kDa hGH-V, a pharmaceutically acceptable excipient and a capsule.

16. A method for treating a patient in need of growth hormone therapy, comprising administering to said patient a 20 kDa hGH-V.

17. The method of claim 16, wherein step of administering includes administering an expression vector capable of producing 20 kDa hGH-V.

18. The method of claim 17, wherein said expression vector is in a host cell.

19. The method of claim 17, wherein said expression vector is in a cell of the patient.

20. A method for reducing lactogenic effects associated with growth hormone therapy, comprising administering to a mammal in need of growth hormone therapy, a composition comprising 20 kDa hGH-V.

21. The method of claim 20, wherein said step of administering includes administering to said mammal, a cell having a replicable vector therein capable of producing 20 kDa hGH-V.
